SEQUENCE LISTING (31 sequences)

SEQ ID NO: 1 - protein, 8 aa, Artificial Sequence
  Description of Artificial Sequence: Synthetic peptide
GFTFDDYA

SEQ ID NO: 2 - protein, 8 aa, Artificial Sequence
  Description of Artificial Sequence: Synthetic peptide
ISWNSGSI

SEQ ID NO: 3 - protein, 16 aa, Artificial Sequence
  Description of Artificial Sequence: Synthetic peptide
AKDSRGYGDYRLGGAY

SEQ ID NO: 4 - protein, 6 aa, Artificial Sequence
  Description of Artificial Sequence: Synthetic peptide
QSVSSN

SEQ ID NO: 5 - protein, 3 aa, Artificial Sequence
  Description of Artificial Sequence: Synthetic peptide
GAS

SEQ ID NO: 6 - protein, 9 aa, Artificial Sequence
  Description of Artificial Sequence: Synthetic peptide
QQYNNWPWT

SEQ ID NO: 7 - protein, 123 aa, Artificial Sequence
  Description of Artificial Sequence: Synthetic polypeptide
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSS

SEQ ID NO: 8 - protein, 107 aa, Artificial Sequence
  Description of Artificial Sequence: Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 9 - protein, 327 aa, Artificial Sequence
  Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 107 aa, Artificial Sequence [sequence omitted]

SEQ ID NO: 11 - protein, 214 aa, Artificial Sequence
  Description of Artificial Sequence: Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 450 aa, Artificial Sequence
  Description of Artificial Sequence: Synthetic polypeptide
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 450 aa, Artificial Sequence
  Description of Artificial Sequence: Synthetic polypeptide
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 327 aa, Artificial Sequence
  Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 15 - protein, 327 aa, Artificial Sequence
  Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 229 aa, Artificial Sequence
  Description of Artificial Sequence: Synthetic polypeptide
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 17 - protein, 229 aa, Artificial Sequence
  Description of Artificial Sequence: Synthetic polypeptide
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 450 aa, Artificial Sequence
  Description of Artificial Sequence: Synthetic polypeptide
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 450 aa, Artificial Sequence
  Description of Artificial Sequence: Synthetic polypeptide
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 20 - protein, 472 aa, Artificial Sequence
  Description of Artificial Sequence: Synthetic polypeptide
EVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSGGGGSEVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 21 - protein, 477 aa, Artificial Sequence
  Description of Artificial Sequence: Synthetic polypeptide
EVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSGGGGSGGGGSEVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 22 - protein, 348 aa, Artificial Sequence
  Description of Artificial Sequence: Synthetic polypeptide
EVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 23 - protein, 5 aa, Artificial Sequence
  Description of Artificial Sequence: Synthetic peptide
GGGGS

SEQ ID NO: 24 - protein, 10 aa, Artificial Sequence
  Description of Artificial Sequence: Synthetic peptide
GGGGSGGGGS

SEQ ID NO: 25 - skipped

SEQ ID NO: 26 - protein, 449 aa, Artificial Sequence
  Description of Artificial Sequence: Synthetic polypeptide
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 27 - protein, 449 aa, Artificial Sequence
  Description of Artificial Sequence: Synthetic polypeptide
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 28 - protein, 471 aa, Artificial Sequence
  Description of Artificial Sequence: Synthetic polypeptide
EVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSGGGGSEVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 29 - protein, 476 aa, Artificial Sequence
  Description of Artificial Sequence: Synthetic polypeptide
EVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSGGGGSGGGGSEVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 30 - protein, 347 aa, Artificial Sequence
  Description of Artificial Sequence: Synthetic polypeptide
EVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 31 - protein, 50 aa, Artificial Sequence
  Description of Artificial Sequence: Synthetic polypeptide
  SITE (1)..(50): This sequence may encompass 1-10 “Gly Gly Gly Gly Ser” repeating units
GGGGSGGGGSGGGGSGGGGSGGGGSGGGGSGGGGSGGGGSGGGGSGGGGS

(19) United States
(12) Patent Application Publication
(10) Pub. No.: US 2021/0403587 A1
(43) Pub. Date: Dec. 30, 2021

(54) METHODS OF TREATING MULTIPLE MYELOMA

(71) Applicant: TeneoBio, Inc., Newark, CA (US)

(72) Inventors: Ben Buelow, Newark, CA (US); Ute Schellenberger, Newark, CA (US)

(21) Appl. No.: 17/244,785

(22) Filed: Apr. 29, 2021

Related U.S. Application Data
(60) Provisional application No. 63/017,597, filed on Apr. 29, 2020.
(60) Provisional application No. 63/073,343, filed on Sep. 1, 2020.
(60) Provisional application No. 63/118,624, filed on Nov. 25, 2020.

(51) Int. Cl. C07K 16/28; A61K 45/06; A61P 35/00

(52) U.S. Cl. CPC C07K 16/2878; A61K 2039/545; A61P 35/00; A61K 45/06

(57) ABSTRACT

Methods of treating multiple myeloma by administering a multispecific antibody to a patient in need are provided. Methods of making such antibodies, and compositions, including pharmaceutical compositions, comprising such antibodies, are also provided.

### CROSS-REFERENCE TO RELATED APPLICATIONS

This application claims priority benefit of the filing date of U.S. Provisional Patent Application Ser. No. 63/017,597, filed on Apr. 29, 2020, as well as U.S. Provisional Patent Application Ser. No. 63/073,343, filed on Sep. 1, 2020, as well as U.S. Provisional Patent Application Ser. No. 63/118,624, filed on Nov. 25, 2020, the disclosures of which applications are incorporated by reference herein in their entireties.

### FIELD OF THE INVENTION

The present invention concerns methods of treating multiple myeloma by administering a multispecific antibody to a patient in need. The invention further concerns methods of making such antibodies, and compositions, including pharmaceutical compositions, comprising such antibodies.

### BACKGROUND

Multiple myeloma (MM) is a plasma cell malignancy and is the second most frequent hematopoietic cancer with an annual incidence of ˜30,000 in the United States (US). The disease is characterized by the proliferation of clonal plasma cells in the bone marrow and is frequently accompanied by the production of a monoclonal immunoglobulin (most frequently IgG, although IgA and light chain-only variants are also common). More than 80% of patients are >60 years of age, with a median age of onset of 68 years; approximately 2% of cases are diagnosed prior to the age of 40 (Jemal A et al., Cancer statistics, 2008; 58(2):71-96; Waxman A J et al., Blood. 2010 Jan. 1; Pulte D et al., The Oncologist. 2011 Oct. 3). The disease primarily localizes to the bones and bone marrow, with resultant cytopenias, bone pain, fractures, infections, hypercalcemia, and renal failure. In addition, serious neurological sequelae can result from pathological fractures in the vertebral bodies. Despite significant morbidity, improvements in myeloma therapy—including proteasome inhibitors, thalidomide derivatives, autologous stem cell therapy, chimeric antigen receptor (CAR) T-cell therapy and anti-CD38 monoclonal antibody (mAb) therapies—have extended median survival >5 years, with some patients surviving ≥10 years (Kumar S K et al., Blood. 2008; 111(5):2516-20; Turesson I et al., Journal of Clinical Oncology. 2010; 28(5):830; Palumbo A et al., New England Journal of Medicine. 2016; 375(8):754-66; Dimopoulos M A et al., New England Journal of Medicine. 2016; 375(14):1319-31; Mikkilineni L et al., Blood. 2017 Jan. 1). Nevertheless, no curative therapy currently exists.

Therapeutic options for advanced MM are limited. Despite the success of therapeutic regimens incorporating PIs, IMiDs, and anti-CD38 mAb therapy, patients that relapse on or are refractory to these therapies have limited therapeutic options (Pick M et al., European Journal of Haematology. 2018; 100(5):494-501). Aspects of the invention include methods of treating this patient population.

### SUMMARY OF THE INVENTION

Aspects of the invention include methods for treating multiple myeloma (MM) in a patient in need, the methods comprising administering to the patient a therapeutically effective amount of TNB-383B according to a 21-day treatment cycle, wherein the therapeutically effective amount of TNB-383B is greater than or equal to 25 μg and less than or equal to a maximum tolerated dose (MTD).

In some embodiments, wherein the treatment cycle is repeated two or more times. In some embodiments, TNB-383B is administered to the patient as a monotherapy. In some embodiments, TNB-383B is administered by an intravenous infusion (IV). In some embodiments, the MM is relapsed MM. In some embodiments, the MM is refractory MM. In some embodiments, the patient has received at least three prior lines of therapy. In some embodiments, one of the prior lines of therapy comprises treatment with a proteasome inhibitor (PI). In some embodiments, one of the prior lines of therapy comprises treatment with an immunomodulatory imide (IMiD). In some embodiments, one of the prior lines of therapy comprises treatment with an anti-CD38 antibody. In some embodiments, the anti-CD38 antibody is a monoclonal antibody. In some embodiments, the anti-CD38 monoclonal antibody is daratumumab.

In some embodiments, the patient is not a candidate for one or more treatment regimens that are known to provide a clinical benefit in MM.

Aspects of the invention include methods for improving an objective response rate (ORR) in a patient diagnosed with MM, the methods comprising administering to the patient a therapeutically effective amount of TNB-383B according to a 21-day treatment cycle, wherein the therapeutically effective amount of TNB-383B is greater than or equal to 25 μg and less than or equal to a maximum tolerated dose (MTD).

Aspects of the invention include methods for improving a clinical benefit rate (CBR) in a patient diagnosed with MM, the methods comprising administering to the patient a therapeutically effective amount of TNB-383B according to a 21-day treatment cycle, wherein the therapeutically effective amount of TNB-383B is greater than or equal to 25 μg and less than or equal to a maximum tolerated dose (MTD).

Aspects of the invention include methods for improving an overall survival (OS) rate in a patient diagnosed with MM, the methods comprising administering to the patient a therapeutically effective amount of TNB-383B according to a 21-day treatment cycle, wherein the therapeutically effective amount of TNB-383B is greater than or equal to 25 μg and less than or equal to a maximum tolerated dose (MTD).

Aspects of the invention include methods for improving a progression free survival (PFS) rate in a patient diagnosed with MM, the methods comprising administering to the patient a therapeutically effective amount of TNB-383B according to a 21-day treatment cycle, wherein the therapeutically effective amount of TNB-383B is greater than or equal to 25 μg and less than or equal to a maximum tolerated dose (MTD).

Aspects of the invention include methods for improving a time to progression (TTP) in a patient diagnosed with MM, the methods comprising administering to the patient a therapeutically effective amount of TNB-383B according to a 21-day treatment cycle, wherein the therapeutically effective amount of TNB-383B is greater than or equal to 25 μg and less than or equal to a maximum tolerated dose (MTD).

Aspects of the invention include methods for improving a time to response (TTR) in a patient diagnosed with MM, the methods comprising administering to the patient a therapeutically effective amount of TNB-383B according to a 21-day treatment cycle, wherein the therapeutically effective amount of TNB-383B is greater than or equal to 25 μg and less than or equal to a maximum tolerated dose (MTD).

Aspects of the invention include methods for improving a duration of objective response (DOR) in a patient diagnosed with MM, the methods comprising administering to the patient a therapeutically effective amount of TNB-383B according to a 21-day treatment cycle, wherein the therapeutically effective amount of TNB-383B is greater than or equal to 25 μg and less than or equal to a maximum tolerated dose (MTD).

In some embodiments, the improvement is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%.

In some embodiments, a treatment cycle is modified to add more time between doses. In some embodiments, a treatment cycle is modified to a 28-day treatment cycle.

In some embodiments, a treatment cycle is modified by consistently eliminating one or more treatment cycles from a dosing regimen. In some embodiments, every third treatment cycle is eliminated from the dosing regimen.

In some embodiments, a treatment cycle is modified to increase a dosing frequency. In some embodiments, a treatment cycle is modified by reducing the time between doses to 14 days.

In some embodiments, a treatment cycle is modified by splitting a dose into multiple portions, and administering each of the multiple portions to the patient on consecutive days.

In some embodiments, a treatment cycle is modified by splitting the dose in half and administering one half of the dose to the patient on each of two consecutive days.

In some embodiments, the methods further comprise premedicating the patient prior to administration of TNB-383B with an agent that reduces a risk or severity of a hypersensitivity reaction. In some embodiments, the agent that reduces the risk or severity of a hypersensitivity reaction is selected from the group consisting of: dexamethasone, diphenhydramine, acetaminophen, ranitidine, any equivalents thereof, or any combination thereof. In some embodiments, the agent that reduces the risk or severity of a hypersensitivity reaction is administered 15 to 60 minutes prior to administration of TNB-383B.

In some embodiments, the maximum tolerated dose (MTD) is selected from the group consisting of: 25 μg, 75 μg, 200 μg, 600 μg, 1,800 μg, 5,400 μg, 10,000 μg, 20,000 μg, 30,000 μg, 40,000 μg, 50,000 μg, 60,000 μg, 70,000 μg, 80,000 μg, 90,000 μg, 100,000 μg, 110,000 μg, 120,000 μg, 130,000 μg, 140,000 μg, 150,000 μg, 160,000 μg, 170,000 μg and 180,000 μg.

Aspects of the invention include methods for treating relapsed or refractory multiple myeloma in a patient in need, the methods comprising administering TNB-383B to the patient at a flat dose ranging from 10 mg to 100 mg, administered once every 3 weeks (21 days), wherein the patient has received at least three prior lines of therapy, including a proteasome inhibitor (PI), an immunomodulatory imide (IMiD) and an anti-CD38 monoclonal antibody (mAb).

Aspects of the invention include methods for treating relapsed or refractory multiple myeloma in a patient in need, the methods comprising administering TNB-383B to the patient at a flat dose of 60 mg, administered once every 3 weeks (21 days), wherein the patient has received at least three prior lines of therapy, including a proteasome inhibitor (PI), an immunomodulatory imide (IMiD) and an anti-CD38 monoclonal antibody (mAb).

These and further aspects will be further explained in the rest of the disclosure, including the Examples.

### BRIEF DESCRIPTION OF THE DRAWINGS

FIG. 1 is a schematic diagram showing an example method of treatment comprising a dose escalation phase (Arm A) and a dose expansion phase (Arm B).

FIG. 2 is a schematic diagram showing an example method of treatment comprising a dose escalation phase (Arm A), and providing additional details regarding treatments for different patient cohorts.

FIG. 3 is a schematic diagram showing suggested treatment guidelines for subject exhibiting signs and/or symptoms of cytokine release syndrome (CRS).

FIG. 4 is a schematic diagram of TNB-383B, which is a three chain antibody like molecule (TCA) comprising one arm comprising a heavy chain/light pair that has binding affinity to CD3, and a second arm comprising a heavy chain-only variable region that has binding affinity to BCMA, in a bivalent configuration.

FIG. 5 provides a summary of subject demographics, subject disposition, and disease characteristics observed for subject enrolled in the clinical study.

FIG. 6 provides a summary of common adverse events observed in the clinical study.

FIG. 7 is a graph and table summarizing CRS response observed from subjects in the clinical study.

FIG. 8 is a graph summarizing subject responses by dose group observed in the clinical study.

FIG. 9 is a swimmer plot showing the trajectory of 27 subject who responded to treatment during the clinical study.

FIG. 10 is a graph showing PK data for TNB-383B observed during the clinical study.

FIG. 11 provides a summary of information relating to a case study of a subject who achieved a VGPR by serology after 1 dose of therapy.

### DETAILED DESCRIPTION OF THE PREFERRED EMBODIMENTS

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless indicated otherwise, antibody residues herein are numbered according to the Kabat numbering system (e.g., Kabat et al., Sequences of Immunological Interest. 5th Ed. Public Health Service, National Institutes of Health, Bethesda, Md. (1991)).

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

All references cited throughout the disclosure, including patent applications and publications, are incorporated by reference herein in their entirety.

### I. Definitions

By “comprising” it is meant that the recited elements are required in the composition/method/kit, but other elements may be included to form the composition/method/kit etc. within the scope of the claim.

By “consisting essentially of”, it is meant a limitation of the scope of composition or method described to the specified materials or steps that do not materially affect the basic and novel characteristic(s) of the subject invention.

By “consisting of”, it is meant the exclusion from the composition, method, or kit of any element, step, or ingredient not specified in the claim.

Antibody residues herein are numbered according to the Kabat numbering system and the EU numbering system. The Kabat numbering system is generally used when referring to a residue in the variable domain (approximately residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies mean residue numbering by the Kabat numbering system. Unless stated otherwise herein, references to residue numbers in the constant domain of antibodies mean residue numbering by the EU numbering system.

Antibodies, also referred to as immunoglobulins, conventionally comprise at least one heavy chain and one light chain, where the amino terminal domain of the heavy and light chains is variable in sequence, hence is commonly referred to as a variable region domain, or a variable heavy (VH) or variable light (VH) domain. The two domains conventionally associate to form a specific binding region, although as will be discussed here, specific binding can also be obtained with heavy chain-only variable sequences, and a variety of non-natural configurations of antibodies are known and used in the art.

A “functional” or “biologically active” antibody or antigen-binding molecule (including heavy chain-only antibodies and multi-specific (e.g., bispecific) three-chain antibody-like molecules (TCAs), described herein) is one capable of exerting one or more of its natural activities in structural, regulatory, biochemical or biophysical events. For example, a functional antibody or other binding molecule, e.g., a TCA, may have the ability to specifically bind an antigen and the binding may in turn elicit or alter a cellular or molecular event such as signal transduction or enzymatic activity. A functional antibody or other binding molecule, e.g., a TCA, may also block ligand activation of a receptor or act as an agonist or antagonist. The capability of an antibody or other binding molecule, e.g., a TCA, to exert one or more of its natural activities depends on several factors, including proper folding and assembly of the polypeptide chains.

The term “antibody” herein is used in the broadest sense and specifically covers monoclonal antibodies, polyclonal antibodies, monomers, dimers, multimers, multispecific antibodies (e.g., bispecific antibodies), heavy chain-only antibodies, three chain antibodies, single chain Fv (scFv), nanobodies, etc., and also includes antibody fragments, so long as they exhibit the desired biological activity (Miller et al (2003) Jour. of Immunology 170:4854-4861). Antibodies may be murine, human, humanized, chimeric, or derived from other species.

The term antibody may reference a full-length heavy chain, a full length light chain, an intact immunoglobulin molecule; or an immunologically active portion of any of these polypeptides, i.e., a polypeptide that comprises an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule, including engineered subclasses with altered Fc portions that provide for reduced or enhanced effector cell activity. Light chains of the subject antibodies can be kappa light chains (Vkappa) or lambda light chains (Vlambda). The immunoglobulins can be derived from any species. In one aspect, the immunoglobulin is of largely human origin.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. Monoclonal antibodies in accordance with the present invention can be made by the hybridoma method first described by Kohler et al. (1975) Nature 256:495, and can also be made via recombinant protein production methods (see, e.g., U.S. Pat. No. 4,816,567), for example.

The term “variable”, as used in connection with antibodies, refers to the fact that certain portions of the antibody variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., residues 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” residues 26-32 (H1), 53-55 (H2) and 96-101 (H3) in the heavy chain variable domain; Chothia and Lesk J. Mol. Biol. 196:901-917 (1987)). In some embodiments, “CDR” means a complementary determining region of an antibody as defined in Lefranc, M P et al., IMGT, the international ImMunoGeneTics database, Nucleic Acids Res., 27:209-212 (1999). “Framework Region” or “FR” residues are those variable domain residues other than the hypervariable region/CDR residues as herein defined.

Exemplary CDR designations are shown herein, however one of skill in the art will understand that a number of definitions of the CDRs are commonly in use, including the Kabat definition (see “Zhao et al. A germline knowledge based computational approach for determining antibody complementarity determining regions.” Mol Immunol. 2010; 47:694-700), which is based on sequence variability and is the most commonly used. The Chothia definition is based on the location of the structural loop regions (Chothia et al. “Conformations of immunoglobulin hypervariable regions.” Nature. 1989; 342:877-883). Alternative CDR definitions of interest include, without limitation, those disclosed by Honegger, “Yet another numbering scheme for immunoglobulin variable domains: an automatic modeling and analysis tool.” J Mol Biol. 2001; 309:657-670; Ofran et al. “Automated identification of complementarity determining regions (CDRs) reveals peculiar characteristics of CDRs and B-cell epitopes.” J Immunol. 2008; 181:6230-6235; Almagro “Identification of differences in the specificity-determining residues of antibodies that recognize antigens of different size: implications for the rational design of antibody repertoires.” J Mol Recognit. 2004; 17:132-143; and Padlan et al. “Identification of specificity-determining residues in antibodies.” Faseb J. 1995; 9:133-139, each of which is herein specifically incorporated by reference.

The terms “heavy chain-only antibody,” and “heavy chain antibody” are used interchangeably herein and refer, in the broadest sense, to antibodies, or more or more portions of an antibody, e.g., one or more arms of an antibody, lacking the light chain of a conventional antibody. The terms specifically include, without limitation, homodimeric antibodies comprising the VH antigen-binding domain and the CH2 and CH3 constant domains, in the absence of the CH1 domain; functional (antigen-binding) variants of such antibodies, soluble VH variants, Ig-NAR comprising a homodimer of one variable domain (V-NAR) and five C-like constant domains (C-NAR) and functional fragments thereof; and soluble single domain antibodies (sUniDabs™). In one embodiment, a heavy chain-only antibody is composed of a variable region antigen-binding domain composed of framework 1, CDR1, framework 2, CDR2, framework 3, CDR3, and framework 4. In another embodiment, a heavy chain-only antibody is composed of an antigen-binding domain, at least part of a hinge region and CH2 and CH3 domains. In another embodiment, a heavy chain-only antibody is composed of an antigen-binding domain, at least part of a hinge region and a CH2 domain. In a further embodiment, a heavy chain-only antibody is composed of an antigen-binding domain, at least part of a hinge region and a CH3 domain Heavy chain-only antibodies in which the CH2 and/or CH3 domain is truncated are also included herein. In a further embodiment, a heavy chain is composed of an antigen binding domain, and at least one CH (CH1, CH2, CH3, or CH4) domain but no hinge region. The heavy chain-only antibody can be in the form of a dimer, in which two heavy chains are disulfide bonded or otherwise, covalently or non-covalently, attached with each other. The heavy chain-only antibody may belong to the IgG subclass, but antibodies belonging to other subclasses, such as IgM, IgA, IgD and IgE subclass, are also included herein. In a particular embodiment, a heavy chain antibody is of the IgG1, IgG2, IgG3, or IgG4 subtype, in particular the IgG1 subtype. In one embodiment, the heavy chain-only antibodies herein are used as a binding (targeting) domain of a chimeric antigen receptor (CAR). The definition specifically includes human heavy chain-only antibodies produced by human immunoglobulin transgenic rats (UniRat™), called UniAbs™. The variable regions (VH) of UniAbs™ are called UniDabs™, and are versatile building blocks that can be linked to Fc regions or serum albumin for the development of novel therapeutics with multi-specificity, increased potency and extended half-life. Since the homodimeric UniAbs™ lack a light chain and thus a VL domain, the antigen is recognized by one single domain, i.e., the variable domain of the heavy chain of a heavy-chain antibody (VH or VHH).

An “intact antibody chain” as used herein is one comprising a full length variable region and a full length constant region (Fc). An intact “conventional” antibody comprises an intact light chain and an intact heavy chain, as well as a light chain constant domain (CL) and heavy chain constant domains, CH1, hinge, CH2 and CH3 for secreted IgG. Other isotypes, such as IgM or IgA may have different CH domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors. Constant region variants include those that alter the effector profile, binding to Fc receptors, and the like.

Depending on the amino acid sequence of the Fc (constant domain) of their heavy chains, antibodies and various antigen-binding proteins can be provided as different classes. There are five major classes of heavy chain Fc regions: IgA, IgD, IgE, IgG, and IgM, and several of these may be further divided into “subclasses” (isotypes), e.g., IgG1, IgG2, IgG3, IgG4, IgA, and IgA2. The Fc constant domains that correspond to the different classes of antibodies may be referenced as α, δ, ε, γ, and μ, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two types, called κ (kappa) and λ (lambda), based on the amino acid sequences of their constant domains. Antibodies in accordance with embodiments of the invention can comprise kappa light chain sequences or lambda light chain sequences.

A “functional Fc region” possesses an “effector function” of a native-sequence Fc region. Non-limiting examples of effector functions include C1q binding; CDC; Fc-receptor binding; ADCC; ADCP; down-regulation of cell-surface receptors (e.g., B-cell receptor), etc. Such effector functions generally require the Fc region to interact with a receptor, e.g., the FcγRI; FcγRIIA; FcγRIIB1; FcγRIIB2; FcγRIIIA; FcγRIIIB receptors, and the low affinity FcRn receptor; and can be assessed using various assays known in the art. A “dead” or “silenced” Fc is one that has been mutated to retain activity with respect to, for example, prolonging serum half-life, but which does not activate a high affinity Fc receptor, or which has a reduced affinity to an Fc receptor.

A “native-sequence Fc region” comprises an amino acid sequence identical to the amino acid sequence of an Fc region found in nature. Native-sequence human Fc regions include, for example, a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, such as 1, 2, 3, 4, 5, 6, 7, 8, 9 or 10 amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

The human IgG4 Fc amino acid sequence (UniProtKB No. P01861) is provided herein as SEQ ID NO: 45. Silenced IgG1 is described, for example, in Boesch, A. W., et al., “Highly parallel characterization of IgG Fc binding interactions.” MAbs, 2014. 6(4): p. 915-27, the disclosure of which is incorporated herein by reference in its entirety.

Other Fc variants are possible, including, without limitation, one in which a region capable of forming a disulfide bond is deleted, or in which certain amino acid residues are eliminated at the N-terminal end of a native Fc, or a methionine residue is added thereto. Thus, in some embodiments, one or more Fc portions of an antibody can comprise one or more mutations in the hinge region to eliminate disulfide bonding. In yet another embodiment, the hinge region of an Fc can be removed entirely. In still another embodiment, an antibody can comprise an Fc variant.

Further, an Fc variant can be constructed to remove or substantially reduce effector functions by substituting (mutating), deleting or adding amino acid residues to effect complement binding or Fc receptor binding. For example, and not limitation, a deletion may occur in a complement-binding site, such as a C1q-binding site. Techniques for preparing such sequence derivatives of the immunoglobulin Fc fragment are disclosed in International Patent Publication Nos. WO 97/34631 and WO 96/32478. In addition, the Fc domain may be modified by phosphorylation, sulfation, acylation, glycosylation, methylation, farnesylation, acetylation, amidation, and the like.

In some embodiments, an antibody comprises a variant human IgG4 CH3 domain sequence comprising a T366W mutation, which can optionally be referred to herein as an IgG4 CH3 knob sequence. In some embodiments, an antibody comprises a variant human IgG4 CH3 domain sequence comprising a T366S mutation, an L368A mutation, and a Y407V mutation, which can optionally be referred to herein as an IgG4 CH3 hole sequence. The IgG4 CH3 mutations described herein can be utilized in any suitable manner so as to place a “knob” on a first heavy chain constant region of a first monomer in an antibody dimer, and a “hole” on a second heavy chain constant region of a second monomer in an antibody dimer, thereby facilitating proper pairing (heterodimerization) of the desired pair of heavy chain polypeptide subunits in the antibody.

In some embodiments, an antibody comprises a heavy chain polypeptide subunit comprising a variant human IgG4 Fc region comprising an S228P mutation, an F234A mutation, an L235A mutation, and a T366W mutation (knob). In some embodiments, and antibody comprises a heavy chain polypeptide subunit comprising a variant human IgG4 Fc region comprising an S228P mutation, an F234A mutation, an L235A mutation, a T366S mutation, an L368A mutation, and a Y407V mutation (hole).

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering of the nucleic acid encoding the antibody. Accordingly, an antibody having an Fc region according to this invention can comprise an antibody with or without K447.

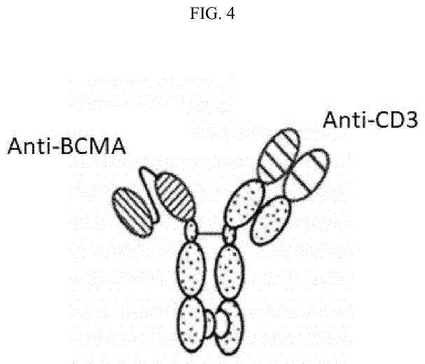

Aspects of the invention include antibodies comprising a heavy chain-only variable region in a monovalent or bivalent configuration. As used herein, the term “monovalent configuration” as used in reference to a heavy chain-only variable region domain means that only one heavy chain-only variable region domain is present, having a single binding site. In contrast, the term “bivalent configuration” as used in reference to a heavy chain-only variable region domain means that two heavy chain-only variable region domains are present (each having a single binding site), and are connected by a linker sequence (see FIG. 4). Non-limiting examples of linker sequences are discussed further herein, and include, without limitation, GS linker sequences of various lengths. When a heavy chain-only variable region is in a bivalent configuration, each of the two heavy chain-only variable region domains can have binding affinity to the same antigen, or to different antigens (e.g., to different epitopes on the same protein; to two different proteins, etc.). However, unless specifically noted otherwise, a heavy chain-only variable region denoted as being in a “bivalent configuration” is understood to contain two identical heavy chain-only variable region domains, connected by a linker sequence, wherein each of the two identical heavy chain-only variable region domains have binding affinity to the same target antigen.

Aspects of the invention include antibodies having multi-specific configurations, which include, without limitation, bispecific, trispecific, etc. A large variety of methods and protein configurations are known and used in bispecific monoclonal antibodies (BsMAB), tri-specific antibodies, etc.

Various methods for the production of multivalent artificial antibodies have been developed by recombinantly fusing variable domains of two or more antibodies. In some embodiments, a first and a second antigen-binding domain on a polypeptide are connected by a polypeptide linker. One non-limiting example of such a polypeptide linker is a GS linker, having an amino acid sequence of four glycine residues, followed by one serine residue, and wherein the sequence is repeated n times, where n is an integer ranging from 1 to about 10, such as 2, 3, 4, 5, 6, 7, 8, or 9. Non-limiting examples of such linkers include GGGGS (SEQ ID NO: 23) (n=1) and GGGGSGGGGS (SEQ ID NO: 24) (n=2). Other suitable linkers can also be used, and are described, for example, in Chen et al., Adv Drug Deliv Rev. 2013 October 15; 65(10): 1357-69, the disclosure of which is incorporated herein by reference in its entirety.

The term “three-chain antibody like molecule” or “TCA” is used herein to refer to antibody-like molecules comprising, consisting essentially of, or consisting of three polypeptide subunits, two of which comprise, consist essentially of, or consist of one heavy and one light chain of a monoclonal antibody, or functional antigen-binding fragments of such antibody chains, comprising an antigen-binding region and at least one CH domain. This heavy chain/light chain pair has binding specificity for a first antigen. The third polypeptide subunit comprises, consists essentially of, or consists of a heavy-chain only antibody comprising an Fc portion comprising CH2 and/or CH3 and/or CH4 domains, in the absence of a CH1 domain, and one or more antigen binding domains (e.g., two antigen binding domains) that binds an epitope of a second antigen or a different epitope of the first antigen, where such binding domain is derived from or has sequence identity with the variable region of an antibody heavy or light chain. Parts of such variable region may be encoded by VH and/or V_{L} gene segments, D and J_{H} gene segments, or J_{L} gene segments. The variable region may be encoded by rearranged V_{H}DJ_{H}, V_{L}DJ_{H}, V_{H}J_{L}, or V_{L}J_{L} gene segments.

A TCA binding compound makes use of a “heavy chain only antibody” or “heavy chain antibody” or “heavy chain polypeptide” which, as used herein, mean a single chain antibody comprising heavy chain constant regions CH2 and/or CH3 and/or CH4 but no CH1 domain. In one embodiment, the heavy chain antibody is composed of an antigen-binding domain, at least part of a hinge region and CH2 and CH3 domains. In another embodiment, the heavy chain antibody is composed of an antigen-binding domain, at least part of a hinge region and a CH2 domain. In a further embodiment, the heavy chain antibody is composed of an antigen-binding domain, at least part of a hinge region and a CH3 domain. Heavy chain antibodies in which the CH2 and/or CH3 domain is truncated are also included herein. In a further embodiment, the heavy chain is composed of an antigen binding domain, and at least one CH (CH1, CH2, CH3, or CH4) domain but no hinge region. The heavy chain only antibody can be in the form of a dimer, in which two heavy chains are disulfide bonded other otherwise covalently or non-covalently attached to each other, and can optionally include an asymmetric interface between one or more of the CH domains to facilitate proper pairing between polypeptide chains. The heavy-chain antibody may belong to the IgG subclass, but antibodies belonging to other subclasses, such as IgM, IgA, IgD and IgE subclass, are also included herein. In a particular embodiment, the heavy chain antibody is of the IgG1, IgG2, IgG3, or IgG4 subtype, in particular the IgG1 subtype or the IgG4 subtype. Non-limiting examples of a TCA binding compound are described in, for example, WO2017/223111 and WO2018/052503, the disclosures of which are incorporated herein by reference in their entirety.

Heavy-chain antibodies constitute about one fourth of the IgG antibodies produced by the camelids, e.g., camels and llamas (Hamers-Casterman C., et al. Nature. 363, 446-448 (1993)). These antibodies are formed by two heavy chains but are devoid of light chains. As a consequence, the variable antigen binding part is referred to as the VHH domain and it represents the smallest naturally occurring, intact, antigen-binding site, being only around 120 amino acids in length (Desmyter, A., et al. J. Biol. Chem. 276, 26285-26290 (2001)). Heavy chain antibodies with a high specificity and affinity can be generated against a variety of antigens through immunization (van der Linden, R. H., et al. Biochim. Biophys. Acta. 1431, 37-46 (1999)) and the VHH portion can be readily cloned and expressed in yeast (Frenken, L. G. J., et al. J. Biotechnol. 78, 11-21 (2000)). Their levels of expression, solubility and stability are significantly higher than those of classical F(ab) or Fv fragments (Ghahroudi, M. A. et al. FEBS Lett. 414, 521-526 (1997)). Sharks have also been shown to have a single VH-like domain in their antibodies, termed VNAR. (Nuttall et al. Eur. J. Biochem. 270, 3543-3554 (2003); Nuttall et al. Function and Bioinformatics 55, 187-197 (2004); Dooley et al., Molecular Immunology 40, 25-33 (2003)).

The term “CD3” refers to the human CD3 protein multi-subunit complex. The CD3 protein multi-subunit complex is composed to 6 distinctive polypeptide chains. These include a CD3γ chain (SwissProt P09693), a CD3δ chain (SwissProtP04234), two CD3e chains (SwissProt P07766), and one CD3 chain homodimer (SwissProt 20963), and which is associated with the T-cell receptor a and chain. The term “CD3” includes any CD3 variant, isoform and species homolog which is naturally expressed by cells (including T-cells) or can be expressed on cells transfected with genes or cDNA encoding those polypeptides, unless noted.

A “BCMA×CD3 antibody” is a multispecific heavy chain-only antibody, such as a bispecific heavy chain-only antibody, which comprises two different antigen-binding regions, one of which binds specifically to the antigen BCMA and one of which binds specifically to CD3.

The term “BCMA” as used herein relates to human B-cell maturation antigen, also known as BCMA, CD269, and TNFRSF17 (UniProt Q02223), which is a member of the tumor necrosis receptor superfamily that is preferentially expressed in differentiated plasma cells. The extracellular domain of human BCMA consists, according to UniProt of amino acids 1-54 (or 5-51).

The term “anti-BCMA heavy chain-only antibody,” and “BCMA heavy chain-only antibody” are used herein to refer to a heavy chain-only antibody as hereinabove defined, immunospecifically binding to BCMA.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Antibodies of the invention include multi-specific antibodies. Multi-specific antibodies have more than one binding specificity. The term “multi-specific” specifically includes “bispecific” and “trispecific,” as well as higher-order independent specific binding affinities, such as higher-order polyepitopic specificity, as well as tetravalent antibodies and antibody fragments. The terms “multi-specific antibody,” “multi-specific heavy chain-only antibody,” “multi-specific heavy chain antibody,” and “multi-specific UniAb™” are used herein in the broadest sense and cover all antibodies with more than one binding specificity.

An “epitope” is the site on the surface of an antigen molecule to which a single antibody molecule binds. Generally an antigen has several or many different epitopes and reacts with many different antibodies. The term specifically includes linear epitopes and conformational epitopes.

“Epitope mapping” is the process of identifying the binding sites, or epitopes, of antibodies on their target antigens. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure.

The term “valent” as used herein refers to a specified number of binding sites in an antibody molecule.

A “monovalent” antibody has one binding site. Thus a monovalent antibody is also monospecific.

A “multi-valent” antibody has two or more binding sites. Thus, the terms “bivalent”, “trivalent”, and “tetravalent” refer to the presence of two binding sites, three binding sites, and four binding sites, respectively. Thus, a bispecific antibody according to the invention is at least bivalent and may be trivalent, tetravalent, or otherwise multi-valent. A bivalent antibody in accordance with embodiments of the invention may have two binding sites to the same epitope (i.e., bivalent, monoparatopic), or to two different epitopes (i.e., bivalent, biparatopic).

A large variety of methods and protein configurations are known and used for the preparation of bispecific monoclonal antibodies (BsMAB), tri-specific antibodies, and the like.

The term “three-chain antibody like molecule” or “TCA” is used herein to refer to antibody-like molecules comprising, consisting essentially of, or consisting of three polypeptide subunits, two of which comprise, consist essentially of, or consist of one heavy chain and one light chain of a monoclonal antibody, or functional antigen-binding fragments of such antibody chains, comprising an antigen-binding region and at least one CH domain. This heavy chain/light chain pair has binding specificity for a first antigen. The third polypeptide subunit comprises, consists essentially of, or consists of a heavy chain-only antibody comprising an Fc portion comprising CH2 and/or CH3 and/or CH4 domains, in the absence of a CH1 domain, and an antigen binding domain that binds an epitope of a second antigen or a different epitope of the first antigen, where such binding domain is derived from or has sequence identity with the variable region of an antibody heavy or light chain. Parts of such variable region may be encoded by V_{H} and/or V_{L} gene segments, D and J_{H} gene segments, or J_{L} gene segments. The variable region may be encoded by rearranged V_{H}DJ_{H}, V_{L}DJ_{H}, V_{H}J_{L}, or V_{L}J_{L} gene segments. A TCA protein makes use of a heavy chain-only antibody as hereinabove defined.

The term “human antibody” is used herein to include antibodies having variable and constant regions derived from human germline immunoglobulin sequences. The human antibodies herein may include amino acid residues not encoded by human germline immunoglobulin sequences, e.g., mutations introduced by random or site-specific mutagenesis in vitro or by somatic mutation in vivo. The term “human antibody” specifically includes heavy chain-only antibodies having human heavy chain variable region sequences, produced by transgenic animals, such as transgenic rats or mice, in particular UniAbs™ produced by UniRats™, as defined above.

By a “chimeric antibody” or a “chimeric immunoglobulin” is meant an immunoglobulin molecule comprising amino acid sequences from at least two different Ig loci, e.g., a transgenic antibody comprising a portion encoded by a human Ig locus and a portion encoded by a rat Ig locus. Chimeric antibodies include transgenic antibodies with non-human Fc-regions or artificial Fc-regions, and human idiotypes. Such immunoglobulins can be isolated from animals of the invention that have been engineered to produce such chimeric antibodies.

As used herein, the term “effector cell” refers to an immune cell which is involved in the effector phase of an immune response, as opposed to the cognitive and activation phases of an immune response. Some effector cells express specific Fc receptors and carry out specific immune functions. In some embodiments, an effector cell such as a natural killer cell is capable of inducing antibody-dependent cellular cytotoxicity (ADCC). For example, monocytes and macrophages, which express FcR, are involved in specific killing of target cells and presenting antigens to other components of the immune system, or binding to cells that present antigens. In some embodiments, an effector cell may phagocytose a target antigen or target cell.

“Human effector cells” are leukocytes which express receptors such as T-cell receptors or FcRs and perform effector functions. Preferably, the cells express at least FcγRIII and perform ADCC effector function. Examples of human leukocytes which mediate ADCC include natural killer (NK) cells, monocytes, cytotoxic T-cells and neutrophils; with NK cells being preferred. The effector cells may be isolated from a native source thereof, e.g., from blood or PBMCs as described herein.

The term “immune cell” is used herein in the broadest sense, including, without limitation, cells of myeloid or lymphoid origin, for instance lymphocytes (such as B-cells and T-cells including cytolytic T-cells (CTLs)), killer cells, natural killer (NK) cells, macrophages, monocytes, eosinophils, polymorphonuclear cells, such as neutrophils, granulocytes, mast cells, and basophils.

Antibody “effector functions” refer to those biological activities attributable to the Fc region (a native sequence Fc region or amino acid sequence variant Fc region) of an antibody. Examples of antibody effector functions include C1q binding; complement dependent cytotoxicity (CDC); Fc receptor binding; antibody-dependent cell-mediated cytotoxicity (ADCC); phagocytosis; down regulation of cell surface receptors (e.g., B-cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g., as described in Gazzano-Santoro et al., J. Immunol. Methods 202:163 (1996), may be performed.

“Binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art. Low-affinity antibodies generally bind antigen slowly and tend to dissociate readily, whereas high-affinity antibodies generally bind antigen faster and tend to remain bound.

As used herein, the “Kd” or “Kd value” refers to a dissociation constant determined by BioLayer Interferometry, using an Octet QK384 instrument (Fortebio Inc., Menlo Park, Calif.) in kinetics mode. For example, anti-mouse Fc sensors are loaded with mouse-Fc fused antigen and then dipped into antibody-containing wells to measure concentration dependent association rates (kon). Antibody dissociation rates (koff) are measured in the final step, where the sensors are dipped into wells containing buffer only. The Kd is the ratio of koff/kon. (For further details see, Concepcion, J, et al., Comb Chem High Throughput Screen, 12(8), 791-800, 2009).

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

A “therapeutically effective amount” is intended for an amount of active agent which is necessary to impart therapeutic benefit to a subject. For example, a “therapeutically effective amount” is an amount which induces, ameliorates or otherwise causes an improvement in the pathological symptoms, disease progression or physiological conditions associated with a disease or which improves resistance to a disorder.

The terms “B-cell neoplasms” or “mature B-cell neoplasms” in the context of the present invention include, but are not limited to, all lymphoid leukemias and lymphomas, chronic lymphocytic leukemia, acute lymphoblastic leukemia, prolymphocytic leukemia, precursor B-lymphoblastic leukemia, hair cell leukemia, small lymphocytic lymphoma, B-cell prolymphocytic lymphoma, B-cell chronic lymphocytic leukemia, mantle cell lymphoma, Burkitt's lymphoma, follicular lymphoma, diffuse large B-cell lymphoma (DLBCL), multiple myeloma, lymphoplasmacytic lymphoma, splenic marginal zone lymphoma, plasma cell neoplasms, such as plasma cell myeloma, plasmacytoma, monoclonal immunoglobulin deposition disease, heavy chain disease, MALT lymphoma, nodal marginal B-cell lymphoma, intravascular large B-cell lymphoma, primary effusion lymphoma, lymphomatoid granulomatosis, non-Hodgkins lymphoma, Hodgkins lymphoma, hairy cell leukemia, primary effusion lymphoma and AIDS-related non-Hodgkins lymphoma.

The term “characterized by expression of BCMA” broadly refers to any disease or disorder in which BCMA expression is associated with or involved with one or more pathological processes that are characteristic of the disease or disorder. Such disorders include, but are not limited to, B-cell neoplasms.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer, individuals with autoimmune diseases, with pathogen infections, and the like. Subjects may be human, but also include other mammals, particularly those mammals useful as laboratory models for human disease, e.g., mouse, rat, etc.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

A “sterile” formulation is aseptic or free or essentially free from all living microorganisms and their spores. A “frozen” formulation is one at a temperature below 0° C.

A “stable” formulation is one in which the protein therein essentially retains its physical stability and/or chemical stability and/or biological activity upon storage. Preferably, the formulation essentially retains its physical and chemical stability, as well as its biological activity upon storage. The storage period is generally selected based on the intended shelf-life of the formulation. Various analytical techniques for measuring protein stability are available in the art and are reviewed in Peptide and Protein Drug Delivery, 247-301. Vincent Lee Ed., Marcel Dekker, Inc., New York, N.Y., Pubs. (1991) and Jones. A. Adv. Drug Delivery Rev. 10: 29-90) (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g., Asn deamidation), oxidation (e.g., Met oxidation), isomerization (e.g., Asp isomeriation), clipping/hydrolysis/fragmentation (e.g., hinge region fragmentation), succinimide formation, unpaired cysteine(s), N-terminal extension, C-terminal processing, glycosylation differences, etc.

Abbreviations used herein include the following: β (Terminal phase elimination rate constant); ADA (Antidrug antibody); ADCC (Antibody-dependent cell cytotoxicity); AE (Adverse event); ALP (Alkaline phosphatase); ALT (Alanine aminotransferase); ANC (Absolute neutrophil count); APRIL (A proliferation inducing ligand); AST (Aspartate aminotransferase); AUC (Area under the concentration-time curve); AUCt (Area under the serum concentration-time curve from time zero to time of last measurable concentration); BCMA (B-cell maturation antigen (also called TNFRSF17)); BUN (Blood urea nitrogen); CAR (Chimeric antigen receptor); CBR (Clinical benefit rate); CI (Confidence interval); CL (Clearance); Cmax (Maximum observed serum concentration); CNS (Central nervous system); CR (Complete response); CRS (Cytokine release syndrome); Css (Steady state concentration); CT (Computed tomography); CTCAE (Common Terminology Criteria for Adverse Events); DLT (Dose limiting toxicity); DNA (Deoxyribonucleic acid); DOR (Duration of Response); ECG (Electrocardiogram); ECHO (Echocardiogram); ECOG (Eastern Cooperative Oncology Group); eCRF (Electronic Case Report Form); EDC (Electronic Data Capture); EE (Efficacy Evaluable); ELISA (Enzyme-linked immunosorbent assay); EOT (End of treatment); FFPE (Formalin-fixed paraffin embedded); FIH (First-in-human); FISH (Fluorescence in situ hybridization); FLC (Free light chain); GCP (Good Clinical Practice); HAV-IgM (Hepatitis A virus immunoglobulin M); HBsAg (Hepatitis B surface antigen); HBV (Hepatitis B virus); HCV (Hepatitis C virus); HCV Ab (Hepatitis C virus antibody); HIV (Human immunodeficiency virus); IB (Investigator's Brochure); ICH (International Conference on Harmonization); IEC (Independent Ethics Committee); IMiD (Immunomodulatory imide); IMWG (International Myeloma Working Group); INR (International normalized ratio); IRB (Institutional Review Board); IV (Intravenous); LDH (Lactate dehydrogenase); mAb (Monoclonal antibody); MABEL (Minimal anticipated biological effect level); MedDRA (Medical Dictionary for Regulatory Activities); MM (Multiple myeloma); MR (Minor response); MRI (Magnetic Resonance Imaging); MTD (Maximum tolerated dose); MUGA (Multiple gated acquisition scan); NCI (National Cancer Institute); MTD (Maximum Tolerated Dose); NCA (Noncompartmental analysis); ORR (Objective response rate); OS (Overall survival); PBMC (Peripheral blood mononuclear cells); PC (Positive control); PET (Positron emission tomography); PI (Proteasome inhibitor); PD (Pharmacodynamic); PK (Pharmacokinetic); PFS (Progression-free Survival); PR (Partial response); PT (Prothrombin time); Q3W (Once every 3 weeks); QTc (QT interval corrected for heart rate); RNA (Ribonucleic acid); RP2D (Recommended phase 2 dose); SAE (Serious adverse event); sCR (Stringent complete response); SIFE (Serum immunofixation electrophoresis); SMG (Safety monitoring group); SPEP (Serum protein electrophoresis); t_{1/2} (Terminal phase elimination half-life); T-BsAbs (T-cell engaging bispecific antibodies); TEAE (Treatment emergent adverse event); Tmax (Time to maximum observed serum concentration); Treg cells (Regulatory T cells); TTP (Time to progression); TTR (Time to response); UIFE (Urine immunofixation electrophoresis); ULN (Upper limit of normal); UPEP (Urine protein electrophoresis); US (United States); V1 (Central compartment volume); VGPR (Very good partial response).

### II. Detailed Description

### TNB-383B

The present invention relates to methods of treating multiple myeloma by administering a bispecific three chain antibody like molecule (TCA) to a patient in need. In a preferred embodiment, a TCA is referred to as TNB-383B, and comprises: an anti-CD3 VH domain that is paired with a light chain variable domain (VL), wherein the VH domain and the VL domain together have binding affinity for CD3; a heavy chain variable domain of a heavy chain-only antibody having binding affinity to BCMA, in a bivalent configuration; and a variant human IgG4 Fc domain comprising a first heavy chain constant region sequence comprising an S228P mutation, an F234A mutation, an L235A mutation, and a T366W mutation (knob), and a second heavy chain constant region sequence comprising an S228P mutation, an F234A mutation, an L235A mutation, a T366S mutation, an L368A mutation, and a Y407V mutation (hole).

In some embodiments, a multi-specific antibody comprises a CD3-binding VH domain that is paired with a light chain variable domain. In certain embodiments, the light chain is a fixed light chain. In some embodiments, the CD3-binding VH domain comprises a CDR1 sequence of SEQ ID NO: 1, a CDR2 sequence of SEQ ID NO: 2, and a CDR3 sequence of SEQ ID NO: 3, in a human VH framework. In some embodiments, the fixed light chain comprises a CDR1 sequence of SEQ ID NO: 4, a CDR2 sequence of SEQ ID NO: 5, and a CDR3 sequence of SEQ ID NO: 6, in a human VL framework. Together, the CD3-binding VH domain and the light chain variable domain have binding affinity for CD3. In some embodiments, a CD3-binding VH domain comprises a heavy chain variable region sequence of SEQ ID NO: 7. In some embodiments, a CD3-binding VH domain comprises a sequence having at least about 80%, at least about 85%, at least about 90%, at least about 95%, or at least about 99% percent identity to the heavy chain variable region sequence of SEQ ID NO: 7. In some embodiments, a fixed light chain comprises a light chain variable region sequence of SEQ ID NO: 8. In some embodiments, a fixed light chain comprises a sequence having at least about 80%, at least about 85%, at least about 90%, at least about 95%, or at least about 99% percent identity to the heavy chain variable region sequence of SEQ ID NO: 8.

Multi-specific antibodies comprising the above-described CD3-binding V_{H} domain and light chain variable domain have advantageous properties, for example, as described in PCT Publication No. WO2018/052503, the disclosure of which is incorporated by reference herein in its entirety.

TABLE 1
Anti-CD3 Heavy and Light Chain CDR1,
CDR2, CDR3 amino acid sequences.

| | SEQ_aa_CDR1 | SEQ_aa_CDR2 | SEQ_aa_CDR3 |
|---|---|---|---|
| Heavy | GFTFDDYA | ISWNSGSI | AKDSRGYGDYRLGGAY |
| Chain | (SEQ ID NO: 1) | (SEQ ID NO: 2) | (SEQ ID NO: 3) |
| Light | QSVSSN | GAS | QQYNNWPWT |
| Chain | (SEQ ID NO: 4) | (SEQ ID NO: 5) | (SEQ ID NO: 6) |

TABLE 2
Anti-CD3 heavy and light chain variable
region amino acid sequences.

| | |
|---|---|
| VH | EVQLVESGGGLVQPGRSLRLSCAASGFTFDDYAMHWVRQAPGKGLE |
| | WVSGISWNSGSIGYADSVKGRFTISRDNAKNSLYLQMNSLRAEDTA |
| | LYYCAKDSRGYGDYRLGGAYWGQGTLVTVSS (SEQ ID NO: 7) |
| VL | EIVMTQSPATLSVSPGERATLSCRASQSVSSNLAWYQQKPGQAPRL |
| | LIYGASTRATGIPARFSGSGSGTEFTLTISSLQSEDFAVYYCQQYN |
| | GNWPWTFGQTKVEIK (SEQ ID NO: 8) |

TABLE 3
Human IgG4 Fc region sequence with
silencing mutations.

| | |
|---|---|
| Human | ASTKGPSVFPLAPCSRSTSESTAALGCLVKDYFPEPVTV |
| IgG4 with | SWNSGALTSGVHTFPAVLQSSGLYSLSSVVTVPSSSLGT |
| silencing | KTYTCNVDHKPSNTKVDKRVESKYGPPCPPCPAPEAAGG |
| mutations | PSVFLFPPKPKDTLMISRTPEVTCVVVDVSQEDPEVQFN |
| (Fc | WYVDGVEVHNAKTKPREEQFNSTYRVVSVLTVLHQDWLN |
| region) | GKEYKCKVSNKGLPSSIEKTISKAKGQPREPQVYTLPPS |
| | QEEMTKNQVSLTCLVKGFYPSDIAVEWESNGQPENNYKT |
| | TPPVLDSDGSFFLYSRLTVDKSRWQEGNVFSCSVMHEAL |
| | HNHYTQKSLSLSLGK (SEQ ID NO: 9) |

TABLE 4
Additional sequences.

| | |
|---|---|
| Anti-CD3 light | RTVAAPSVFIFPPSDEQLKSGTASVVCLLNNFYPREAKVQWKVDNAL |
| chain constant | QSGNSQESVTEQDSKDSTYSLSSTLTLSKADYEKHKVYACEVTHQGL |
| region sequence | SSPVTKSFNRGEC (SEQ ID NO: 10) |
| (kappa light | |
| chain) | |
| Anti-CD3 full | EIVMTQSPATLSVSPGERATLSCRASQSVSSNLAWYQQKPGQAPRLL |
| length light | IYGASTRATGIPARFSGSGSGTEFTLTISSLQSEDFAVYYCQQYNNW |
| chain (VL + | PWTFGQGTKVEIKRTVAAPSVFIFPPSDEQLKSGTASVVCLLNNFYP |
| kappa CL) | REAKVQWKVDNALQSGNSQESVTEQDSKDSTYSLSSTLTLSKADYEK |
| | HKVYACEVTHQGLSSPVTKSFNRGEC (SEQ ID NO: 11) |
| Anti-CD3 heavy | EVQLVESGGGLVQPGRSLRLSCAASGFTFDDYAMHWVRQAPGKGLEW |
| chain VH F2B | VSGISWNSGSIGYADSVKGRFTISRDNAKNSLYLQMNSLRAEDTALY |
| (+wt IgG4 Fc) | YCAKDSRGYGDYRLGGAYWGQGTLVTVSSASTKGPSVFPLAPCSRST |
| | SESTAALGCLVKDYFPEPVTVSWNSGALTSGVHTFPAVLQSSGLYSL |
| | SSVVTVPSSSLGTKTYTCNVDHKPSNTKVDKRVESKYGPPCPSCPAP |
| | EFLGGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSNK |
| | GLPSSIEKTISKAKGQPREPQVYTLPPSQEEMTKNQVSLTCLVKGFY |
| | PSDIAVEWESNGQPENNYKTTPPVLDSDGSFFLYSRLTVDKSRWQEG |
| | NVFSCSVMHEALHNHYTQKSLSLSLGK (SEQ ID NO: 12) |
| Anti-CD3 heavy | EVQLVESGGGLVQPGRSLRLSCAASGFTFDDYAMHWVRQAPGKGLEW |
| chain VH F2B | VSGISWNSGSIGYADSVKGRFTISRDNAKNSLYLQMNSLRAEDTALY |
| (+silenced | YCAKDSRGYGDYRLGGAYWGQGTLVTVSSASTKGPSVFPLAPCSRST |
| IgG4 Fc) | SESTAALGCLVKDYFPEPVTVSWNSGALTSGVHTFPAVLQSSGLYSL |
| | SSVVTVPSSSLGTKTYTCNVDHKPSNTKVDKRVESKYGPPCP [symbol] CPAP |
| | E [symbol] GGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSNK |
| | GLPSSIEKTISKAKGQPREPQVYTLPPSQEEMTKNQVSLTCLVKGFY |
| | PSDIAVEWESNGQPENNYKTTPPVLDSDGSFFLYSRLTVDKSRWQEG |
| | NVFSCSVMHEALHNHYTQKSLSLSLGK (SEQ ID NO: 13) |
| Silenced IgG4 | ASTKGPSVFPLAPCSRSTSESTAALGCLVKDYFPEPVTVSWNSGALT |
| (CH1-hinge- | SGVHTFPAVLQSSGLYSLSSVVTVPSSSLGTKTYTCNVDHKPSNTKV |
| CH2-CH3; hole | DKRVESKYGPPCP [symbol] CPAPE [symbol] GGPSVFLFPPKPKDTLMISRTPEVTC |
| (S228P, F234A, | VVVDVSQEDPEVQFNWYVDGVEVHNAKTKPREEQFNSTYRVVSVLTV |
| L235A; T366S, | LHQDWLNGKEYKCKVSNKGLPSSIEKTISKAKGQPREPQVYTLPPSQ |
| L368A, Y407V)) | EEMTKNQVSL [symbol] C [symbol] VKGFYPSDIAVEWESNGQPENNYKTTPPVLDSDG |
| | SFFL [symbol] SRLTVDKSRWQEGNVFSCSVMHEALHNHYTQKSLSLSLGK |
| | (SEQ ID NO: 14) |
| Silenced IgG4 | ASTKGPSVFPLAPCSRSTSESTAALGCLVKDYFPEPVTVSWNSGALT |
| (CH1-hinge- | SGVHTFPAVLQSSGLYSLSSVVTVPSSSLGTKTYTCNVDHKPSNTKV |
| CH2-CH3; knob | RDKVESKYGPPCP [symbol] CPAPE [symbol] GGPSVFLFPPKPKDTLMISRTPEVTC |
| (S228P, F234A, | VVVDVSQEDPEVQFNWYVDGVEVHNAKTKPREEQFNSTYRVVSVLTV |
| L235A; T366W)) | LHQDWLNGKEYKCKVSNKGLPSSIEKTISKAKGQPREPQVYTLPPSQ |
| | EEMTKNQVSL [symbol] CLVKGFYPSDIAVEWESNGQPENNYKTTPPVLDSDG |
| | SFFLYSRLTVDKSRWQEGNVFSCSVMHEALHNHYTQKSLSLSLGK |
| | (SEQ ID NO: 15) |
| Silenced IgG4 | ESKYGPPCP [symbol] CPAPE [symbol] GGPSVFLFPPKPKDTLMISRTPEVTCVVVD |
| (hinge-CH2- | VSQEDPEVQFNWYVDGVEVHNAKTKPREEQFNSTYRVVSVLTVLHQD |
| CH3; hole | WLNGKEYKCKVSNKGLPSSIEKTISKAKGQPREPQVYTLPPSQEEMT |
| (S228P, F234A, | KNQVSL [symbol] C [symbol] VKGFYPSDIAVEWESNGQPENNYKTTPPVLDSDGSFFL |
| L235A; T366S, | [symbol] SRLTVDKSRWQEGNVFSCSVMHEALHNHYTQKSLSLSLGK (SEQ |
| L368A, Y407V)) | ID NO: 16) |
| Silenced IgG4 | ESKYGPPCP [symbol] CPAPE [symbol] GGPSVFLFPPKPKDTLMISRTPEVTCVVVD |
| (hinge-CH2- | VSQEDPEVQFNWYVDGVEVHNAKTKPREEQFNSTYRVVSVLTVLHQD |
| CH3; knob | WLNGKEYKCKVSNKGLPSSIEKTISKAKGQPREPQVYTLPPSQEEMT |
| (S228P, F234A, | KNQVSL [symbol] CLVKGFYPSDIAVEWESNGQPENNYKTTPPVLDSDGSFFL |
| L235A; T366W)) | YSRLTVDKSRWQEGNVFSCSVMHEALHNHYTQKSLSLSLGK (SEQ |
| | ID NO: 17) |
| Anti-CD3 full | EVQLVESGGGLVQPGRSLRLSCAASGFTFDDYAMHWVRQAPGKGLEW |
| length heavy | VSGISWNSGSIGYADSVKGRFTISRDNAKNSLYLQMNSLRAEDTALY |
| chain (VH | YCAKDSRGYGDYRLGGAYWGQGTLVTVSSASTKGPSVFPLAPCSRST |
| F2B + silenced | SESTAALGCLVKDYFPEPVTVSWNSGALTSGVHTFPAVLQSSGLYSL |
| IgG4 Fc + knob | SSVVTVPSSSLGTKTYTCNVDHKPSNTKVDKRVESKYGPPCP [symbol] CPAP |
| (S228P, F234A, | E [symbol] GGPSVFLFPPKPKDTLMISRTPEVTCVVVDVSQEDPEVQFNWYV |
| L235A; T366W)), | DGVEVHNAKTKPREEQFNSTYRVVSVLTVLHQDWLNGKEYKCKVSNK |
| with C-terminal | GLPSSIEKTISKAKGQPREPQVYTLPPSQEEMTKNQVSL [symbol] CLVKGFY |
| Lysine (K) | PSDIAVEWESNGQPENNYKTTPPVLDSDGSFFLYSRLTVDKSRWQEG |
| | NVFSCSVMHEALHNHYTQKSLSLSLGK (SEQ ID NO: 18) |
| Anti-CD3 full | EVQLVESGGGLVQPGRSLRLSCAASGFTFDDYAMHWVRQAPGKGLEW |
| length heavy | VSGISWNSGSIGYADSVKGRFTISRDNAKNSLYLQMNSLRAEDTALY |
| chain (VH F2B + | YCAKDSRGYGDYRLGGAYWGQGTLVTVSSASTKGPSVFPLAPCSRST |
| silenced IgG4 | SESTAALGCLVKDYFPEPVTVSWNSGALTSGVHTFPAVLQSSGLYSL |
| Fc + knob | SSVVTVPSSSLGTKTYTCNVDHKPSNTKVDKRVESKYGPPCP [symbol] CPAP |
| (S228P, F234A, | E [symbol] GGPSVFLFPPKPKDTLMISRTPEVTCVVVDVSQEDPEVQFNWYV |
| L235A; T366W)), | DGVEVHNAKTKPREEQFNSTYRVVSVLTVLHQDWLNGKEYKCKVSNK |
| without | GLPSSIEKTISKAKGQPREPQVYTLPPSQEEMTKNQVSL [symbol] CLVKGFY |
| C-terminal | PSDIAVEWESNGQPENNYKTTPPVLDSDGSFFLYSRLTVDKSRWQEG |
| Lysine (K) | NVFSCSVMHEALHNHYTQKSLSLSLG (SEQ ID NO: 26) |
| Anti-CD3 | EVQLVESGGGLVQPGRSLRLSCAASGFTFDDYAMHWVRQAPGKGLEW |
| full length | VSGISWNSGSIGYADSVKGRFTISRDNAKNSLYLQMNSLRAEDTALY |
| heavy chain | YCAKDSRGYGDYRLGGAYWGQGTLVTVSSASTKGPSVFPLAPCSRST |
| (VH F2B + | SESTAALGCLVKDYFPEPVTVSWNSGALTSGVHTFPAVLQSSGLYSL |
| silenced IgG4 | SSVVTVPSSSLGTKTYTCNVDHKPSNTKVDKRVESKYGPPCP [symbol] CPAP |
| Fc + hole | E [symbol] GGPSVFLFPPKPKDTLMISRTPEVTCVVVDVSQEDPEVQFNWYV |
| (S228P, F234A, | DGVEVHNAKTKPREEQFNSTYRVVSVLTVLHQDWLNGKEYKCKVSNK |
| L235A; T366S, | GLPSSIEKTISKAKGQPREPQVYTLPPSQEEMTKNQVSL [symbol] C [symbol] VKGFY |
| L368A, Y407V)), | PSDIAVEWESNGQPENNYKTTPPVLDSDGSFFL [symbol] SRLTVDKSRWQEG |
| with | NVFSCSVMHEALHNHYTQKSLSLSLGK (SEQ ID NO: 19) |
| C-terminal | |
| Lysine (K) | |
| Anti-CD3 | EVQLVESGGGLVQPGRSLRLSCAASGFTFDDYAMHWVRQAPGKGLEW |
| full length | VSGISWNSGSIGYADSVKGRFTISRDNAKNSLYLQMNSLRAEDTALY |
| heavy chain | YCAKDSRGYGDYRLGGAYWGQGTLVTVSSASTKGPSVFPLAPCSRST |
| (VH F2B + | SESTAALGCLVKDYFPEPVTVSWNSGALTSGVHTFPAVLQSSGLYSL |
| silenced IgG4 | SSVVTVPSSSLGTKTYTCNVDHKPSNTKVDKRVESKYGPPCP [symbol] CPAP |
| Fc + hole | E [symbol] GGPSVFLFPPKPKDTLMISRTPEVTCVVVDVSQEDPEVQFNWYV |
| (S228P, F234A, | DGVEVHNAKTKPREEQFNSTYRVVSVLTVLHQDWLNGKEYKCKVSNK |
| L235A; T366S, | GLPSSIEKTISKAKGQPREPQVYTLPPSQEEMTKNQVSL [symbol] C [symbol] VKGFY |
| L368A, Y407V)), | PSDIAVEWESNGQPENNYKTTPPVLDSDGSFFL [symbol] SRLTVDKSRWQEG |
| without | NVFSCSVMHEALHNHYTQKSLSLSLG (SEQ ID NO: 27) |
| C-terminal | |
| Lysine (K) | |

TABLE 5
Additional sequences.

| | |
|---|---|
| Silenced IgG4 | ESKYGPPCP [symbol] CPAPE [symbol] GGPSVFLFPPKPKDTLMISRTPEVTCVVVD |
| (hinge-CH2- | VSQEDPEVQFNWYVDGVEVHNAKTKPREEQFNSTYRVVSVLTVLHQD |
| CH3; hole | WLNGKEYKCKVSNKGLPSSIEKTISKAKGQPREPQVYTLPPSQEEMT |
| (S228P, F234A, | KNQVSL [symbol] C [symbol] VKGFYPSDIAVEWESNGQPENNYKTTPPVLDSDGSFFL |
| L235A; T366S, | [symbol] SRLTVDKSRWQEGNVFSCSVMHEALHNHYTQKSLSLSLGK (SEQ |
| L368A, Y407V)) | ID NO: 16) |
| Silenced IgG4 | ESKYGPPCP [symbol] CPAPE [symbol] GGPSVFLFPPKPKDTLMISRTPEVTCVVVD |
| (hinge-CH2- | VSQEDPEVQFNWYVDGVEVHNAKTKPREEQFNSTYRVVSVLTVLHQD |
| CH3; knob | WLNGKEYKCKVSNKGLPSSIEKTISKAKGQPREPQVYTLPPSQEEMT |
| (S228P, F234A, | KNQVSL [symbol] CLVKGFYPSDIAVEWESNGQPENNYKTTPPVLDSDGSFFL |
| L235A; T366W)) | YSRLTVDKSRWQEGNVFSCSVMHEALHNHYTQKSLSLSLGK (SEQ |
| | ID NO: 17) |
| BCMA bivalent | EVQLLESGGGLVQPGGSLRLSCAASGFTVSSYGMSWVRQAPGKGPEW |
| heavy chain | VSGIRGSDGSTYYADSVKGRFTISRDNSKNTLYLQMNSLRAEDTAVY |
| (TNB-383B w | YCAKQGENDGPFDHRGQGTLVTVSSGGGGSEVQLLESGGGLVQPGGS |
| GS1) + silenced | LRLSCAASGFTVSSYGMSWVRQAPGKGPEWVSGIRGSDGSTYYADSV |
| IgG4 Fc hinge | KGRFTISRDNSKNTLYLQMNSLRAEDTAVYYCAKQGENDGPFDHRGQ |
| CH2 CH3, hole | GTLVTVSSESKYGPPCP [symbol] CPAPE [symbol] GGPSVFLFPPKPKDTLMISRTP |
| (S228P, F234A, | EVTCVVVDVSQEDPEVQFNWYVDGVEVHNAKTKPREEQFNSTYRVVS |
| L235A, T366S, | VLTVLHQDWLNGKEYKCKVSNKGLPSSIEKTISKAKGQPREPQVYTL |
| L368A, Y407V, | PPSQEEMTKNQVSL [symbol] C [symbol] VKGFYPSDIAVEWESNGQPENNYKTTPPVL |
| with C-terminal | DSDGSFFL [symbol] SRLTVDKSRWQEGNVFSCSVMHEALHNHYTQKSLSLSL |
| Lysine (K) | GK (SEQ ID NO: 20) |
| BCMA bivalent | EVQLLESGGGLVQPGGSLRLSCAASGFTVSSYGMSWVRQAPGKGPEW |
| heavy chain | VSGIRGSDGSTYYADSVKGRFTISRDNSKNTLYLQMNSLRAEDTAVY |
| (TNB-383B w | YCAKQGENDGPFDHRGQGTLVTVSSGGGGSEVQLLESGGGLVQPGGS |
| GS1) + silenced | LRLSCAASGFTVSSYGMSWVRQAPGKGPEWVSGIRGSDGSTYYADSV |
| IgG4 Fc hinge | KGRFTISRDNSKNTLYLQMNSLRAEDTAVYYCAKQGENDGPFDHRGQ |
| CH2 CH3, hole | GTLVTVSSESKYGPPC [symbol] PCPAPE [symbol] GGPSVFLFPPKPKDTLMISRTP |
| (S228P, F234A, | EVTCVVVDVSQEDPEVQFNWYVDGVEVHNAKTKPREEQFNSTYRVVS |
| L235A, T366S, | VLTVLHQDWLNGKEYKCKVSNKGLPSSIEKTISKAKGQPREPQVYTL |
| L368A, Y407V, | PPSQEEMTKNQVSL [symbol] C [symbol] VKGFYPSDIAVEWESNGQPENNYKTTPPVL |
| without | DSDGSFFL [symbol] SRLTVDKSRWQEGNVFSCSVMHEALHNHYTQKSLSLSL |
| C-terminal | G (SEQ ID NO: 28) |
| Lysine (K) | |
| BCMA bivalent | EVQLLESGGGLVQPGGSLRLSCAASGFTVSSYGMSWVRQAPGKGPEW |
| heavy chain | VSGIRGSDGSTYYADSVKGRFTISRDNSKNTLYLQMNSLRAEDTAVY |
| (TNB-383B w | YCAKQGENDGPFDHRGQGTLVTVSSGGGGSGGGGSEVQLLESGGGLV |
| G52) + silenced | QPGGSLRLSCAASGFTVSSYGMSWVRQAPGKGPEWVSGIRGSDGSTY |
| IgG4 Fc hinge | YADSVKGRFTISRDNSKNTLYLQMNSLRAEDTAVYYCAKQGENDGPF |
| CH2 CH3, hole | DHRGQGTLVTVSSESKYGPPCP [symbol] CPAPE [symbol] GGPSVFLFPPKPKDTLM |
| (S228P, F234A, | ISRTPEVTCVVVDVSQEDPEVQFNWYVDGVEVHNAKTKPREEQFNST |
| L235A, T366S, | YRVVSVLTVLHQDWLNGKEYKCKVSNKGLPSSIEKTISKAKGQPREP |
| L368A, Y407V, | QVYTLPPSQEEMTKNQVSL [symbol] C [symbol] VKGFYPSDIAVEWESNGQPENNYKT |
| with C-terminal | TPPVLDSDGSFFL [symbol] SRLTVDKSRWQEGNVFSCSVMHEALHNHYTQKS |
| Lysine (K) | LSLSLGK (SEQ ID NO: 21) |
| BCMA bivalent | EVQLLESGGGLVQPGGSLRLSCAASGFTVSSYGMSWVRQAPGKGPEW |
| heavy chain | VSGIRGSDGSTYYADSVKGRFTISRDNSKNTLYLQMNSLRAEDTAVY |
| (TNB-383B w | YCAKQGENDGPFDHRGQGTLVTVSSGGGGSGGGGSEVQLLESGGGLV |
| GS2) + silenced | QPGGSLRLSCAASGFTVSSYGMSWVRQAPGKGPEWVSGIRGSDGSTY |
| IgG4 Fc hinge | YADSVKGRFTISRDNSKNTLYLQMNSLRAEDTAVYYCAKQGENDGPF |
| CH2 CH3, hole | DHRGQGTLVTVSSESKYGPPCP [symbol] CPAPE [symbol] GGPSVFLFPPKPKDTLM |
| (S228P, F234A, | ISRTPEVTCVVVDVSQEDPEVQFNWYVDGVEVHNAKTKPREEQFNST |
| L235A, T366S, | YRVVSVLTVLHQDWLNGKEYKCKVSNKGLPSSIEKTISKAKGQPREP |
| L368A, Y407V, | QVYTLPPSQEEMTKNQVSL [symbol] C [symbol] VKGFYPSDIAVEWESNGQPENNYKT |
| without | TPPVLDSDGSFFL [symbol] SRLTVDKSRWQEGNVFSCSVMHEALHNHYTQKS |
| C-terminal | LSLSLG (SEQ ID NO: 29) |
| Lysine (K) | |
| BCMA monovalent | EVQLLESGGGLVQPGGSLRLSCAASGFTVSSYGMSWVRQAPGKGPEW |
| heavy chain | VSGIRGSDGSTYYADSVKGRFTISRDNSKNTLYLQMNSLRAEDTAVY |
| (TNB-383B) + | YCAKQGENDGPFDHRGQGTLVTVSSESKYGPPCP [symbol] CPAPE [symbol] GGPSV |
| silenced IgG4 | FLFPPKPKDTLMISRTPEVTCVVVDVSQEDPEVQFNWYVDGVEVHNA |
| Fc hinge CH2 | KTKPREEQFNSTYRVVSVLTVLHQDWLNGKEYKCKVSNKGLPSSIEK |
| CH3, hole | TISKAKGQPREPQVYTLPPSQEEMTKNQVSL [symbol] C [symbol] VKGFYPSDIAVEW |
| (S228P, F234A | ESNGQPENNYKTTPPVLDSDGSFFL [symbol] SRLTVDKSRWQEGNVFSCSVM |
| L235A T366S, | HEALHNHYTQKSLSLSLGK (SEQ ID NO: 22) |
| L368A, Y407V, | |
| with C-terminal | |
| Lysine (K) | |
| BCMA monovalent | EVQLLESGGGLVQPGGSLRLSCAASGFTVSSYGMSWVRQAPGKGPEW |
| heavy chain | VSGIRGSDGSTYYADSVKGRFTISRDNSKNTLYLQMNSLRAEDTAVY |
| (TNB-383B) + | YCAKQGENDGPFDHRGQGTLVTVSSESKYGPPCP [symbol] CPAPE [symbol] GGPSV |
| silenced IgG4 | FLFPPKPKDTLMISRTPEVTCVVVDVSQEDPEVQFNWYVDGVEVHNA |
| Fc hinge CH2 | KTKPREEQFNSTYRVVSVLTVLHQDWLNGKEYKCKVSNKGLPSSIEK |
| CH3, hole F234A | TISKAKGQPREPQVYTLPPSQEEMTKNQVSL [symbol] C [symbol] VKGFYPSDIAVEW |
| L235A (S228P, | ESNGQPENNYKTTPPVLDSDGSFFL [symbol] SRLTVDKSRWQEGNVFSCSVM |
| T366S, L368A, | HEALHNHYTQKSLSLSLG (SEQ ID NO: 30) |
| Y407V, without | |
| C-terminal | |
| Lysine (K) | |

In some embodiments, bispecific or multi-specific antibodies are provided, which may have any of the configurations discussed herein, including, without limitation, a bispecific three-chain antibody like molecule. In some embodiments, a bispecific antibody can comprise at least one heavy chain variable region having binding specificity for BCMA, and at least one heavy chain variable region having binding specificity for a different protein, e.g., CD3. In some embodiments, a bispecific antibody can comprise a heavy chain/light chain pair that has binding specificity for a first antigen, and a heavy chain from a heavy chain-only antibody, comprising an Fc portion comprising CH2 and/or CH3 and/or CH4 domains, in the absence of a CH1 domain, and an antigen binding domain that binds an epitope of a second antigen or a different epitope of the first antigen, in a monovalent or bivalent configuration. In one particular embodiment, a bispecific antibody comprises a heavy chain/light chain pair that has binding specificity for an antigen on an effector cell (e.g., a CD3 protein on a T-cell), and a heavy chain from a heavy chain-only antibody comprising an antigen-binding domain that has binding specificity for BCMA, in a monovalent or bivalent configuration.

In some embodiments, where an antibody of the invention is a bispecific antibody, one arm of the antibody (one binding moiety, or one binding unit) is specific for human BCMA, while the other arm may be specific for target cells, tumor-associated antigens, targeting antigens, e.g., integrins, etc., pathogen antigens, checkpoint proteins, and the like. Target cells specifically include cancer cells. In some embodiments, one arm of the antibody (one binding moiety, or one binding unit) is specific for human BCMA, while the other arm is specific for CD3.

In some embodiments, an antibody comprises an anti-CD3 light chain polypeptide comprising the sequence of SEQ ID NO: 8 linked to the sequence of SEQ ID NO: 10, an anti-CD3 heavy chain polypeptide comprising the sequence of any one of SEQ ID NOs: 12, 13, 14, 15, 18, or 19, and an anti-BCMA heavy chain polypeptide comprising the sequence of any one of SEQ ID NOs: 20, 21 or 22. In one preferred embodiment, an antibody is a TCA comprising a first polypeptide comprising SEQ ID NO: 11, a second polypeptide comprising SEQ ID NO: 18, and a third polypeptide comprising SEQ ID NO: 20, 21 or 22. In one preferred embodiment, an antibody is a TCA comprises a first polypeptide comprising SEQ ID NO: 11, a second polypeptide comprising SEQ ID NO: 18, and a third polypeptide comprising SEQ ID NO: 20. In one preferred embodiment, an antibody is a TCA consisting of a first polypeptide consisting of SEQ ID NO: 11, a second polypeptide consisting of SEQ ID NO: 18, and a third polypeptide consisting of SEQ ID NO: 21. In one preferred embodiment, an antibody is a TCA comprising a first polypeptide comprising SEQ ID NO: 11, a second polypeptide comprising SEQ ID NO: 18, and a third polypeptide comprising SEQ ID NO: 22. In one preferred embodiment, an antibody is a TCA consisting of a first polypeptide consisting of SEQ ID NO: 11, a second polypeptide consisting of SEQ ID NO: 18, and a third polypeptide consisting of SEQ ID NO: 20. In one preferred embodiment, TNB-383B consists of a first polypeptide consisting of SEQ ID NO: 11, a second polypeptide consisting of SEQ ID NO: 18, and a third polypeptide consisting of SEQ ID NO: 20.

### Preparation of Antibodies

The multispecific antibodies of the present invention can be prepared by methods known in the art. In a preferred embodiment, the heavy chain antibodies herein are produced by transgenic animals, including transgenic mice and rats, preferably rats, in which the endogenous immunoglobulin genes are knocked out or disabled. In a preferred embodiment, the heavy chain antibodies herein are produced in UniRat™. UniRat™ have their endogenous immunoglobulin genes silenced and use a human immunoglobulin heavy-chain translocus to express a diverse, naturally optimized repertoire of fully human HCAbs. While endogenous immunoglobulin loci in rats can be knocked out or silenced using a variety of technologies, in UniRat™ the zinc-finger (endo)nuclease (ZNF) technology was used to inactivate the endogenous rat heavy chain J-locus, light chain Cκ locus and light chain Cλ locus. ZNF constructs for microinjection into oocytes can produce IgH and IgL knock out (KO) lines. For details see, e.g., Geurts et al., 2009, Science 325:433. Characterization of Ig heavy chain knockout rats has been reported by Menoret et al., 2010, Eur. J. Immunol. 40:2932-2941. Advantages of the ZNF technology are that non-homologous end joining to silence a gene or locus via deletions up to several kb can also provide a target site for homologous integration (Cui et al., 2011, Nat Biotechnol 29:64-67). Human heavy chain antibodies produced in UniRat™ are called UniAbs™ and can bind epitopes that cannot be attacked with conventional antibodies. Their high specificity, affinity, and small size make them ideal for mono- and poly-specific applications.

In addition to UniAbs™, specifically included herein are heavy chain-only antibodies lacking the camelid VHH framework and mutations, and their functional V_{H} regions. Such heavy chain-only antibodies can, for example, be produced in transgenic rats or mice which comprise fully human heavy chain-only gene loci as described, e.g., in WO2006/008548, but other transgenic mammals, such as rabbit, guinea pig, rat can also be used, rats and mice being preferred. Heavy chain-only antibodies, including their VHH or VH functional fragments, can also be produced by recombinant DNA technology, by expression of the encoding nucleic acid in a suitable eukaryotic or prokaryotic host, including, for example, mammalian cells (e.g., CHO cells), E. coli or yeast.

Domains of heavy chain-only antibodies combine advantages of antibodies and small molecule drugs: can be mono- or multi-valent; have low toxicity; and are cost-effective to manufacture. Due to their small size, these domains are easy to administer, including oral or topical administration, are characterized by high stability, including gastrointestinal stability; and their half-life can be tailored to the desired use or indication. In addition, VH and VHH domains of HCAbs can be manufactured in a cost effective manner.

In a particular embodiment, the heavy chain antibodies of the present invention, including UniAbs™, have the native amino acid residue at the first position of the FR4 region (amino acid position 101 according to the Kabat numbering system), substituted by another amino acid residue, which is capable of disrupting a surface-exposed hydrophobic patch comprising or associated with the native amino acid residue at that position. Such hydrophobic patches are normally buried in the interface with the antibody light chain constant region but become surface exposed in HCAbs and are, at least partially, for the unwanted aggregation and light chain association of HCAbs. The substituted amino acid residue preferably is charged, and more preferably is positively charged, such as lysine (Lys, K), arginine (Arg, R) or histidine (His, H), preferably arginine (R). In a preferred embodiment the heavy chain-only antibodies derived from the transgenic animals contain a Trp to Arg mutation at position 101. The resultant HCAbs preferably have high antigen-binding affinity and solubility under physiological conditions in the absence of aggregation.

As part of the present invention, human heavy chain antibodies with unique sequences from UniRat™ animals (UniAb™) were identified that bind human CD3 and BCMA in ELISA protein and cell-binding assays. The identified heavy chain variable region (VH) sequences (see, e.g., Tables 2, 4 and 5) are positive for protein binding and/or for binding to cells expressing the target protein (e.g., CD3 or BCMA), and are all negative for binding to cells that do not express the target protein.

Heavy chain antibodies binding to non-overlapping epitopes on a target protein, e.g., UniAbs™ can be identified by competition binding assays, such as enzyme-linked immunoassays (ELISA assays) or flow cytometric competitive binding assays. For example, one can use competition between known antibodies binding to the target antigen and the antibody of interest. By using this approach, one can divide a set of antibodies into those that compete with the reference antibody and those that do not. The non-competing antibodies are identified as binding to a distinct epitope that does not overlap with the epitope bound by the reference antibody. Often, one antibody is immobilized, the antigen is bound, and a second, labeled (e.g., biotinylated) antibody is tested in an ELISA assay for ability to bind the captured antigen. This can be performed also by using surface plasmon resonance (SPR) platforms, including ProteOn XPR36 (BioRad, Inc), Biacore 2000 and Biacore T200 (GE Healthcare Life Sciences), and MX96 SPR imager (Ibis technologies B.V.), as well as on biolayer interferometry platforms, such as Octet Red384 and Octet HTX (ForteBio, Pall Inc). For further details see the examples herein.

Typically, an antibody “competes” with a reference antibody if it causes about 15-100% reduction in the binding of the reference antibody to the target antigen, as determined by standard techniques, such as by the competition binding assays described above. In various embodiments, the relative inhibition is at least about 15%, at least about 20%, at least about 25%, at least about 30%, at least about 35%, at least about 40%, at least about 45%, at least about 50% at least about 55%, at least about 60%, at least about 65%, at least about 70%, at least about 75%, at least about 80%, at least about 85%, at least about 90%, at least about 95% or higher.

### Methods of Use

The antibodies and pharmaceutical compositions described herein can be used for the treatment of diseases and conditions characterized by the expression of a target protein (e.g., CD3, BCMA), including, without limitation, the conditions and diseases described further herein. In preferred embodiments, the antibodies and pharmaceutical compositions described herein can be used for the treatment of diseases and conditions characterized by the expression of BCMA.

The pharmaceutical compositions herein comprising anti-BCMA antibodies can be used for the treatment of B-cell related disorders, including B-cell and plasma cell malignancies and autoimmune disorders characterized by the expression or overexpression of BCMA.

Such B-cell related disorders include B-cell and plasma cell malignancies and autoimmune disorders, including, without limitation, multiple myeloma, plasmacytoma, Hodgkins' lymphoma, follicular lymphomas, small non-cleaved cell lymphomas, endemic Burkitt's lymphoma, sporadic Burkitt's lymphoma, marginal zone lymphoma, extranodal mucosa-associated lymphoid tissue lymphoma, nodal monocytoid B-cell lymphoma, splenic lymphoma, mantle cell lymphoma, large cell lymphoma, diffuse mixed cell lymphoma, immunoblastic lymphoma, primary mediastinal B-cell lymphoma, pulmonary B-cell angiocentric lymphoma, small lymphocytic lymphoma, B-cell proliferations of uncertain malignant potential, lymphomatoid granulomatosis, post-transplant lymphoproliferative disorder, an immunoregulatory disorder, rheumatoid arthritis, myasthenia gravis, idiopathic thrombocytopenia purpura, anti-phospholipid syndrome, Chagas' disease, Grave's disease, Wegener's granulomatosis, poly-arteritis nodosa, Sjogren's syndrome, pemphigus vulgaris, scleroderma, multiple sclerosis, anti-phospholipid syndrome, ANCA associated vasculitis, Goodpasture's disease, Kawasaki disease, autoimmune hemolytic anemia, and rapidly progressive glomerulonephritis, heavy-chain disease, primary or immunocyte-associated amyloidosis, or monoclonal gammopathy.

The plasma cell disorders characterized by the expression of BCMA include Multiple Myeloma (MM). MM is a B-cell malignancy characterized by a monoclonal expansion and accumulation of abnormal plasma cells in the bone marrow compartment. Current therapies for MM often cause remissions, but nearly all patients eventually relapse and die. There is substantial evidence of an immune-mediated elimination of myeloma cells in the setting of allogeneic hematopoietic stem cell transplantation; however, the toxicity of this approach is high, and few patients are cured. Although some monoclonal antibodies have shown promise for treating MM in preclinical studies and early clinical trials, consistent clinical efficacy of any monoclonal antibody therapy for MM has not been conclusively demonstrated. There is therefore a great need for new therapies, including immunotherapies for MM (see, e.g., Carpenter et al., Clin Cancer Res 2013, 19(8):2048-2060).

Overexpression or activation of BCMA by its proliferation-inducing ligand, APRIL it known to promote human Multiple Myeloma (MM) progression in vivo. BCMA has also been shown to promote in vivo growth of xenografted MM cells harboring p53 mutation in mice. Since activity of the APRIL/BCMA pathway plays a central role in MM pathogenesis and drug resistance via bidirectional interactions between tumor cells and their supporting bone marrow microenvironment, BCMA has been identified as a target for the treatment of MM. For further details see, e.g., Yu-Tsu Tai et al., Blood 2016; 127(25):3225-3236.

Another B-cell disorder involving plasma cells i.e. expressing BCMA is systemic lupus erythematosus (SLE), also known as lupus. SLE is a systemic, autoimmune disease that can affect any part of the body and is represented with the immune system attacking the body's own cells and tissue, resulting in chronic inflammation and tissue damage. It is a Type III hypersensitivity reaction in which antibody-immune complexes precipitate and cause a further immune response (Inaki & Lee, Nat Rev Rheumatol 2010; 6: 326-337).

The anti-BCMA heavy chain-only antibodies (UniAb) of the present invention can be used to develop therapeutic agents for the treatment of MM, SLE, and other B-cell disorders or plasma cell disorders characterized by the expression of BCMA, such as those listed above. In particular, the anti-BCMA heavy chain-only antibodies (UniAb) of the present invention are candidates for the treatment of MM, alone or in combination with other MM treatments.

Effective doses of the compositions of the present invention for the treatment of disease vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g., companion animals such as dogs, cats, horses, etc., laboratory mammals such as rabbits, mice, rats, etc., and the like. Treatment dosages can be titrated to optimize safety and efficacy.

Typically, compositions are prepared as injectables, either as liquid solutions or suspensions; solid forms suitable for solution in, or suspension in, liquid vehicles prior to injection can also be prepared. The pharmaceutical compositions herein are suitable for intravenous or subcutaneous administration, directly or after reconstitution of solid (e.g., lyophilized) compositions. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the antibodies and antibody structures described herein can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in formulating a dosage range that is not toxic for use in humans. The dosage of the antibodies described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The compositions for administration will commonly comprise an antibody or other agent (e.g., another ablative agent) dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Aspects of the invention include methods of treating relapsed or refractory multiple myeloma by administering TNB-383B as a fourth line therapy to patients who have previously been exposed to treatment with a proteasome inhibitor (PI), an immunomodulatory imide (IMiD) and an anti-CD38 monoclonal antibody (mAb). In some embodiments, TNB-383B is administered as a fourth line therapy for relapsed or refractory multiple myeloma at a flat dose ranging from 10 mg to 100 mg, administered once every three weeks, in patients who have previously been treated with a proteasome inhibitor (PI), an immunomodulatory imide (IMiD) and an anti-CD38 monoclonal antibody (mAb).

In some embodiments, a method comprises administering TNB-383B as a fourth line therapy at a flat dose of 10 mg, administered once every 3 weeks (21 days), to a patient with relapsed or refractory multiple myeloma who has received at least three prior lines of therapy, including a proteasome inhibitor (PI), an immunomodulatory imide (IMiD) and an anti-CD38 monoclonal antibody (mAb).

In some embodiments, a method comprises administering TNB-383B as a fourth line therapy at a flat dose of 20 mg, administered once every 3 weeks (21 days), to a patient with relapsed or refractory multiple myeloma who has received at least three prior lines of therapy, including a proteasome inhibitor (PI), an immunomodulatory imide (IMiD) and an anti-CD38 monoclonal antibody (mAb).

In some embodiments, a method comprises administering TNB-383B as a fourth line therapy at a flat dose of 30 mg, administered once every 3 weeks (21 days), to a patient with relapsed or refractory multiple myeloma who has received at least three prior lines of therapy, including a proteasome inhibitor (PI), an immunomodulatory imide (IMiD) and an anti-CD38 monoclonal antibody (mAb).

In some embodiments, a method comprises administering TNB-383B as a fourth line therapy at a flat dose of 40 mg, administered once every 3 weeks (21 days), to a patient with relapsed or refractory multiple myeloma who has received at least three prior lines of therapy, including a proteasome inhibitor (PI), an immunomodulatory imide (IMiD) and an anti-CD38 monoclonal antibody (mAb).

In some embodiments, a method comprises administering TNB-383B as a fourth line therapy at a flat dose of 50 mg, administered once every 3 weeks (21 days), to a patient with relapsed or refractory multiple myeloma who has received at least three prior lines of therapy, including a proteasome inhibitor (PI), an immunomodulatory imide (IMiD) and an anti-CD38 monoclonal antibody (mAb).

In some embodiments, a method comprises administering TNB-383B as a fourth line therapy at a flat dose of 60 mg, administered once every 3 weeks (21 days), to a patient with relapsed or refractory multiple myeloma who has received at least three prior lines of therapy, including a proteasome inhibitor (PI), an immunomodulatory imide (IMiD) and an anti-CD38 monoclonal antibody (mAb).

In some embodiments, a method comprises administering TNB-383B as a fourth line therapy at a flat dose of 70 mg, administered once every 3 weeks (21 days), to a patient with relapsed or refractory multiple myeloma who has received at least three prior lines of therapy, including a proteasome inhibitor (PI), an immunomodulatory imide (IMiD) and an anti-CD38 monoclonal antibody (mAb).

In some embodiments, a method comprises administering TNB-383B as a fourth line therapy at a flat dose of 80 mg, administered once every 3 weeks (21 days), to a patient with relapsed or refractory multiple myeloma who has received at least three prior lines of therapy, including a proteasome inhibitor (PI), an immunomodulatory imide (IMiD) and an anti-CD38 monoclonal antibody (mAb).

In some embodiments, a method comprises administering TNB-383B as a fourth line therapy at a flat dose of 90 mg, administered once every 3 weeks (21 days), to a patient with relapsed or refractory multiple myeloma who has received at least three prior lines of therapy, including a proteasome inhibitor (PI), an immunomodulatory imide (IMiD) and an anti-CD38 monoclonal antibody (mAb).

In some embodiments, a method comprises administering TNB-383B as a fourth line therapy at a flat dose of 100 mg, administered once every 3 weeks (21 days), to a patient with relapsed or refractory multiple myeloma who has received at least three prior lines of therapy, including a proteasome inhibitor (PI), an immunomodulatory imide (IMiD) and an anti-CD38 monoclonal antibody (mAb).

In some embodiments, the methods further comprise combination therapy, wherein one or more additional multiple myeloma treatments, e.g., one of more chemotherapeutic drugs, is administered to the patient in combination with TNB-383B, as described above.

Also within the scope of the invention are kits comprising the active agents and formulations thereof, of the invention and instructions for use. The kit can further contain a least one additional reagent, e.g., a chemotherapeutic drug, etc. Kits typically include a label indicating the intended use of the contents of the kit. The term “label” as used herein includes any writing, or recorded material supplied on or with a kit, or which otherwise accompanies a kit.

Aspects of the invention include methods for evaluating the safety, pharmacokinetic (PK), pharmacodynamic (PD) and clinical activity of TNB-383B in subjects with relapsed or refractory multiple myeloma (MM) who have received at least 3 prior lines of therapy, including a proteasome inhibitor (PI), an immunomodulatory imide (IMiD) and an anti-CD38 mAb (e.g., daratumumab). “Line/regimen of therapy” is defined as a course of therapy (comprising at least 1 cycle) not interrupted by progressive disease, except in circumstances where the drug is not tolerated due to toxicity.

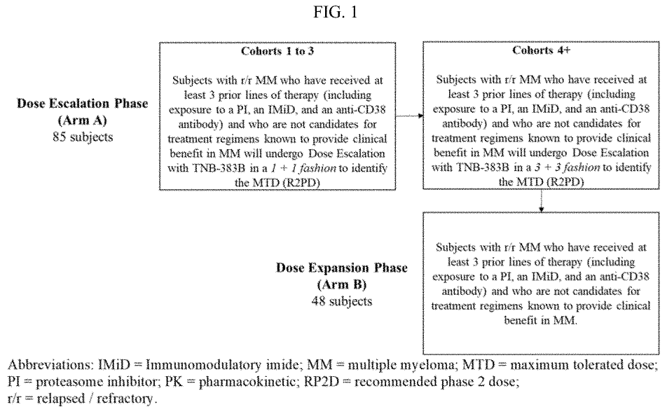

Aspects of the invention include methods that involve a Monotherapy Dose Escalation Protocol (Arm A) and a Monotherapy Dose Expansion Protocol (Arm B) (FIG. 1).

### Monotherapy Dose Escalation (Arm A):

In some embodiments, the methods involve evaluating the safety, tolerability, PK and PD profiles of single-agent TNB-383B therapy administered once every three weeks (Q3W, 21-day cycle), in patients with relapsed/refractory MM who have received at least 3 prior lines of therapy, including a PI, an IMiD and an anti-CD38 mAb (e.g., daratumumab).

In some embodiments, the methods involve administering a single dose of TNB-383B on a 21-day cycle for at least one cycle. In some embodiments, a TNB-383B dose is selected from the group consisting of: 25 μg, 75 μg, 200 μg, 600 μg, 1,800 μg, 5,400 μg, 10,000 μg, 20,000 μg, 30,000 μg, 40,000 μg, 50,000 μg, 60,000 μg, 70,000 μg, 80,000 μg, 90,000 μg, 100,000 μg, 110,000 μg, 120,000 μg, 130,000 μg, 140,000 μg, 150,000 μg, 160,000 μg, 170,000 μg and 180,000 μg.

In some embodiments, the methods involve a dose escalation that begins with a Q3W dosing schedule at a first dose, and then the patient is administered an increased dose. In certain embodiments, dose escalation is contingent upon no evidence of drug related toxicity (e.g., a Grade 2+ toxicity) at the first dose in the patient. In some embodiments, the methods involve an alternative dosing regimen. For example, in some embodiments, dosing may be switched to a different frequency (e.g., once every 4 weeks) or some cycles may be consistently eliminated from a dosing schedule (e.g., every scheduled 3^{rd} cycle will be dropped). If the dosing schedule is switched to occur more frequently, no dose modification should result in a predicted steady state concentration (CSS) or Cmax greater than those identified for the next lower dose level. Split dosing of the designated dose on 2 consecutive days (to reduce cytokine release) may also be implemented based on data review. In some embodiments, dose limiting toxicity (DLT) criteria are used to make decisions regarding dose escalation. The maximum tolerated dose (MTD) is defined as the highest dose level at which <2 of 6 evaluable subjects experience a DLT. In some embodiments, dose escalation is based on clinically significant toxicity, DLT events, PK and PD findings (when available), and is implemented by safety monitors. In some embodiments, an MTD is selected from the group consisting of: 25 μg, 75 μg, 200 μg, 600 μg, 1,800 μg, 5,400 μg, 10,000 μg, 20,000 μg, 30,000 μg, 40,000 μg, 50,000 μg, 60,000 μg, 70,000 μg, 80,000 μg, 90,000 μg, 100,000 μg, 110,000 μg, 120,000 μg, 130,000 μg, 140,000 μg, 150,000 μg, 160,000 μg, 170,000 μg and 180,000 μg.

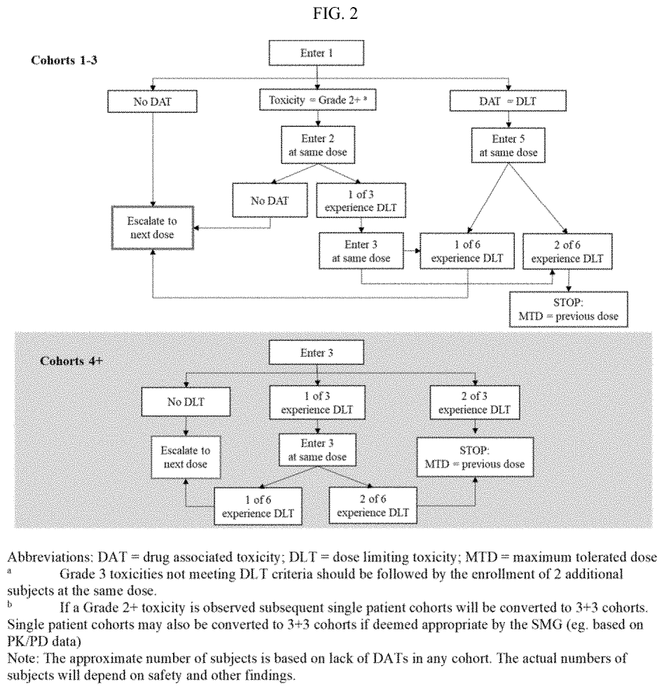

In some embodiments, dose escalation to subsequent (higher) dose levels is implemented if multiple (e.g., the first 3) DLT evaluable subjects (or the first evaluable subject at the lowest 3 dose levels) in a given cohort complete a safety assessment during the first cycle without experiencing a DLT. In some embodiments, if 1 subject at a given dose level experiences a DLT, that same dose level is expanded to 6 subjects (or 3 subjects in the case of Grade 2+ toxicity not meeting DLT criteria in Cohorts 1-3; FIG. 2). In some embodiments, subsequent single patient cohorts are converted to 3+3 cohorts if a Grade 2+ toxicity is observed. In a 3+3 study design, 3 patients are enrolled in a cohort corresponding to given dose level. In some embodiments, single patient cohorts are converted to 3+3 cohorts if deemed appropriate by safety monitors (e.g., based on PK/PD data). In some embodiments, if 2 of 6 subjects experience a DLT, dose escalation will stop because the MTD will have been exceeded. In such embodiments, the previous dose level may be declared the MTD, or a new intermediate dose, lower than the intolerable dose, may be evaluated based on review of the data. In some embodiments, if 1 of 6 evaluable subjects experiences a DLT, escalation to the next proposed dose level proceeds. Given the algorithm above, the probability of escalation to the next higher dose is approximately 90% if true DLT rate is 10%, 70% if true DLT rate is 20%, 43% if true DLT rate is 33%, and 17% if true DLT rate is 50%. In some embodiments, if the MTD is not reached, or the RP2D is not identified, then higher dose levels (at increases of no more than 50%) may be evaluated after review of all currently available safety and PK/PD data.

A non-limiting example of a dose escalation scheme for Arm A is shown in Table 6, and non-limiting examples of dose escalation guidelines are described in Table 7.

TABLE 6
Monotherapy Dose Escalation: Arm A

| | | Proposed (Actual) | |
|---|---|---|---|
| | | Number | Proposed Additional |
| Cohort^{b} | Dose (μg Q3W) | of Subjects^{a} | Subjects^{c} |
| 1 | 25 | 1 (3) | |
| 2 | 75 | 1 (3) | |
| 3 | 200 | 3 (3) | |
| 4 | 600 | 3 (3) | |
| 5 | 1800 | 3 (3) | |
| 6 | 5400 | 3 (6) | |
| 7 | 10000 | 3 (9) | |
| 8 | 20000 | 3 (7) | |
| 9 | 30000 | 3 (6) | |
| 10 | 40000 | 3 (6) | |
| 11 | 60000 | 3 (6) | |
| 12 | 50000 | 3 (3) | |
| 13 | 90000 | 3 | 18^{d} |
| 14 | 120000 | 3 | |
| 15 | 180000 | 3 | |

^{a}The approximate number of subjects is based on lack of dose associated toxicities in any cohort. The actual numbers of subjects will depend on safety and other findings. The MTD cohort will be expanded to 6 subjects to further characterize the cohort.
^{b}Dose de-escalation from any dose level (except starting 25 μg) may occur to refine the determination of the MTD and/or RP2D.
^{c}If the available data during SMG review of cohort N as equivocal regarding further escalation (e.g. based on occurrence of non-DLT AEs, plateaued efficacy, suspected RP2D), cohort N and/or cohort N − 1 may be expanded up to a maximum of 9 patients each at the discretion of the SMG.
^{d}Approximately 18 additional subjects may be enrolled in Cohorts 13 through 15, up to a maximum of 9 subjects total per cohort.

TABLE 7
Monotherapy Dose Escalation Guidelines

| | |
|---|---|
| [symbol] | [symbol] |
| 1 of 1 (Grade 2+) | Enroll a total of 3 subjects in |
| | current dose level. |
| 1 of 1 (DLT) | Enroll a total of 6 subjects in |
| | current dose level. |
| 0 of 3 | Begin enrollment in the next dose |
| | level. |
| 1 of 3 (DLT) | Enroll a total of 6 subjects in |
| | current dose level. |
| 1 of 6 (DLT) | Begin enrollment in the next dose |
| | level. |
| 2 of 6 | Dose escalation stops and previous |
| (or ≥ 33.3%, DLT) | dose determined as MTD or new |
| | intermediate dose evaluated. |

Abbreviations:
DLT = dose limiting toxicity;
MTD = maximum tolerated dose [symbol]
indicates data missing or illegible when filed

[symbol]

In some embodiments, after a safety review of cycle 1 for a cohort N is complete, and if that dose is deemed safe (e.g., the safety monitors endorse further dose escalation or the dose for cohort N is determined to be the RP2D), any subjects that remain under treatment at a dose of TNB-383B lower than that assigned to cohort N may subsequently be treated at the dose assigned to cohort N (e.g., when the safety review for cohort 5 is complete and the decision to escalate to cohort 6 has been made, any patients still under treatment from cohorts 1-4 may have their dose increased to the dose corresponding to cohort 5). In some embodiments, subjects must have previously received at least 2 cycles of TNB-383B, without any drug-related toxicities leading to a dose reduction, to be eligible for such a dose escalation.

### Monotherapy Dose Expansion—Arm B:

In some embodiments, the methods involve evaluating the MTD (or RP2D) of TNB-383B monotherapy in patients with relapsed or refractory MM who have received at least 3 prior lines of therapy (including exposure to a PI, an IMiD and an anti-CD38 mAb (e.g., daratumumab)). In some embodiments, dose expansion is initiated once the MTD (or RP2D) has been selected based on data from a Monotherapy Dose Escalation Phase (Arm A). In some embodiments, the MTD (or RP2D) and dosing frequency for Arm B are chosen based on safety, tolerability, and PK/PD data collected during the dose escalation portion of the study. All subjects within the observation window (e.g., subjects either under treatment with TNB-383B or status-post last dose but within the 90-day follow-up period and not having initiated a new line of therapy) are evaluated for safety and tolerability of the regimen, PK/PD profile, and preliminary evidence of activity each time 6 additional subjects are accrued to the study. After the Monotherapy Dose Expansion Phase has been initiated, if the dose level/frequency is modified, no dose modification should result in a predicted CSS or Cmax greater than those identified for the previously selected MTD/RP2D.

In some embodiments, patients undergo screening procedures within 28 days prior to initial drug administration. Adult subjects who meet the inclusion criteria and who do not meet any of the exclusion criteria are eligible for treatment.

### Inclusion Criteria:

1. Subject must be ≤18 years of age.
2. Three or more prior lines of therapy with exposure to a PI, and IMiD, and an anti-CD38 antibody (e.g., daratumumab). In order to be eligible for this study subjects must not be candidates for treatment regimens known to provide clinical benefit in MM.
3. Subject has an Eastern Cooperative Oncology Group (ECOG) Performance Status of ≤2.
4. Subject is capable of understanding and complying with parameters as outlined in the protocol and able to sign informed consent.
5. Subject must have adequate bone marrow function, defined as: absolute neutrophil count (ANC) ≥1000/mm³; platelets ≥50,000/mm³; hemoglobin ≥8.0 g/dL. Transfusion and/or growth factor support is permitted prior to assessment, but neutrophils, platelets, and hemoglobin must be stable for at least 72 hours after transfusion and/or growth factor administration prior to screening for the subject to be eligible.
6. Subject must have an eGFR ≥30 mL/min as estimated by the MDRD formula.
7. Subject must have total bilirubin ≤1.5× upper limit of normal (ULN; except if the subject has a known diagnosis of Gilbert's syndrome, in which case bilirubin must be ≤3×ULN), aspartate aminotransferase (AST) and alanine aminotransferase (ALT)≤3×ULN.
8. Serum calcium (corrected for albumin) at or below the ULN range (subject may enroll in the setting of hypercalcemia IF hypercalcemia resolves with standard treatment by Cycle 1, Day 1) prior to study therapy initiation.
9. If female, subject must be either postmenopausal (for at least 12 consecutive months), OR permanently surgically sterile, OR for women of childbearing potential practicing at least 1 protocol specified method of female birth control AND one method of male birth control (vasectomy or condoms), starting at Cycle 1, Day 1 through at least 6 months after the last dose of study drug.
10. Measurable Disease: Subject has a diagnosis of MM and documented prior treatment with a PI, an IMiD, and an anti-CD38 mAb therapy (e.g., daratumumab) as part of 3 or more lines of therapy. There is no maximum number of prior regimens and prior bone marrow transplant is acceptable if subject is >12 weeks (autologous) or >1 year (allogeneic) status-post transplantation. In order to be eligible for this study subjects must not be candidates for treatment regimens known to provide clinical benefit in MM.

Measurable disease is defined as at least 1 of the following:
Serum M-protein ≥0.5 g/dL (≥5 g/L)
Urine M-protein ≥200 mg/24h
Serum free light chain (FLC) assay: Involved FLC level ≥10 mg/dl (≥100 mg/L) and an abnormal serum FLC ratio (<0.26 or >1.65).
11. Subject has confirmed evidence of relapse/progression from the immediately prior MM therapy, or subject is relapsed/refractory to the immediate prior MM therapy. (‘Relapsed/refractory’ is defined as subjects with a history of minimal response [MR] or better response to prior therapy, now with disease progression within 60 days of the last therapy. ‘Relapse’ is defined as previously treated myeloma that progresses and requires initiation of salvage therapy, without meeting International Myeloma Working Group [IMWG] uniform response criteria for relapsed/refractory.)
12. Subject has adequate archival tumor bone marrow sample if available or consents to a fresh pre-treatment bone marrow tumor biopsy.

### Exclusion Criteria

A subject will not be eligible for treatment if he/she meets any of the following criteria:
1. Subject has been diagnosed or treated for another malignancy within 3 years of enrollment, with the exception of basal cell or squamous cell carcinoma of the skin, in situ malignancy, low-risk prostate carcinoma after curative therapy, or complete resection/curative therapy of an advanced malignancy.
2. Subject has a history of CNS involvement by their myeloma.
3. Subject has a history of ≥Grade 3 peripheral neuropathy.
4. Subject has a history of plasma cell leukemia, POEMS syndrome, or amyloidosis.
5. Subject has received another investigational drug within 21 days of enrollment.
6. Subject has ever received BCMA-targeted therapy. Subjects who have received targeted therapy against non-BCMA targets will not be excluded.
7. Subject has received a peripheral autologous stem cell transplant within 12 weeks, or an allogeneic transplant within 1 year of the first dose of study drug treatment.
8. Subject has any medical or psychiatric condition which in the opinion of the investigator or Study Medical Monitor places the subject at an unacceptably high risk for toxicities, could interfere with successful or safe delivery of therapy, or could interfere with evaluation of the investigational product or interpretation of subject safety or study results. Examples include history of significant mucosal/internal bleeding, major psychiatric illness, drug abuse (including active alcoholism), or known allergy or hypersensitivity to components of the study drug formulation.
9. Subject has received any therapy to treat cancer (including radiation, chemotherapy, biologics, cellular therapies, and/or steroids at doses >20 mg) or undergone a major surgical procedure within 21 days, or within 5 half-lives of an anticancer drug, prior to the first dose of study treatment, whichever is shorter.
10. Subject has known active infection requiring parenteral anti-infective treatment. Upon completion of antibiotics and resolution of symptoms, the subject is considered eligible for the study from an infection standpoint.
11. Confirmed positive test results for human immunodeficiency virus (HIV), or subjects with chronic or active hepatitis B virus (HBV) or hepatitis C virus (HCV). Subjects who have a history of HBV or HCV who have documented cures (HBV: HBsAg negative; HCV: undetectable HCV ribonucleic acid (RNA) 24 weeks after the end of treatment) may be enrolled.
12. Major cardiac abnormalities, such as but not limited to the following: uncontrolled angina or unstable life-threatening arrhythmias, history of myocardial infarction <12 weeks before Screening, Class ≥3 New York Heart Association congestive heart failure, severe cardiac insufficiency, or persistent QTc prolongation (>480 msec, QTc Fridericia).
13. Subject has unresolved AEs ≥Grade 2 (National Cancer Institute (NCI) Common Terminology Criteria for Adverse Events (CTCAE] v5.0) from prior anticancer therapy except for:
   Alopecia.
   Peripheral neuropathy (peripheral neuropathy ≥Grade 3 will be excluded).
   Anemia or thrombocytopenia (thrombocytopenia must be Grade 4 to trigger exclusion, Grade 3 with symptoms or bleeding, respectively, or return within 72 hours despite transfusion support).
   Subjects with irreversible toxicity that is not reasonably expected to be exacerbated by any of the investigational products may be included (e.g., hearing loss) after consultation with the Study Medical Monitor.

In some embodiments, TNB-383B is initially administered as an IV infusion Q3W, where 1 cycle of treatment is 21 days. In some embodiments, a 25 μg starting dose of TNB-383B is administered as an IV infusion (Q3W) in the Monotherapy Dose Escalation Phase (Arm A) and escalated to a projected maximum dose of 180,000 μg in subsequent cohorts (Table 6). In some embodiments, in Arm B, all subjects receive TNB-383B at the MTD and/or RP2D. Subjects may continue to receive TNB-383B as long as they do not meet any of the criteria for subject discontinuation.

In some embodiments, subjects are premedicated with dexamethasone (5-20 mg IV) or equivalent, diphenhydramine (25 to 50 mg IV) or equivalent (e.g., Cetirizine 10 mg PO×1), acetaminophen 650 to 1000 mg PO and ranitidine 150 mg PO/IV or equivalent 15 to 60 minutes prior to TNB-383B infusion to reduce the risk and severity of hypersensitivity reactions commonly observed with mAb therapy.

In some embodiments, the first TNB-383B infusion is given over 2 hours (±10 minutes). If no infusion reactions occur during the first dose of TNB-383B, the duration of infusion for subsequent doses of TNB-383B may be shortened in some embodiments. In some embodiments, subjects are monitored for 4 hours after the first infusion, and for 2 hours after each subsequent infusion. For clarity, the 4 hours of close observation post-infusion in Cycle 1 should occur during the Subject's hospitalization after the first dose of TNB-383B. In some embodiments, a subject is hospitalized for a total of 48 hours, from Day 1 to Day 3, following the first dose of TNB-383B.

### Preparation/Reconstitution of Dosage Form:

In some embodiments, a TNB-383B drug product (active) is provided as a solution in vials, formulated at 2 mg/mL with 10 mL of extractable volume of drug product per vial, and administered by IV infusion. In some embodiments, TNB-383B is diluted in 2 steps. The first dilution step reduces the strength of TNB-383B 100-fold to 20 μg/mL using a non-DEHP 50 mL IV bag, provided with the kit. The second dilution step requires dose-dependent transfer of a specified volume of pre-diluted TNB-383B into a non-DEHP-containing 250 mL IV bag, also provided with the kit. The final concentrations are chosen to achieve the desired dose to be administered. In some embodiments, the diluent for each dilution step is saline with IV stabilizer solution (IVSS) added prior to the addition of active TNB-383B drug product. The IVSS is provided with each kit and consists of a 20 mL glass vial with 20 mL extractable volume. The IVSS vial is formulated at 12.5× strength, with a working strength of 1× in the IV bag.

In some embodiments, TNB-383B is prepared in a single dilution step, with dose-dependent volumes of TNB-383B transferred from drug product vial directly into a 250 mL non-DEHP-containing IV bag, provided with each kit. The diluent is saline with IVSS added prior to the addition of active TNB-383B drug product.

In some embodiments, total storage time (including infusion time) of the IV Bag containing the final dilution of TNB-383B at controlled room temperature (20-25° C.) does not exceed 6 hours (or 12 hours at 2-8° C.) to minimize degradation of the Drug Product and the risk of microbiological contamination. In some embodiments, the storage time is modified/updated as additional sterility/stability data become available.

In some embodiments, the total volume administered at each dose is 250 mL. Infusion rates are controlled with infusion pumps and their respective DEHP-free infusion sets containing inline filters.

In some embodiments, TNB-383B drug product vials are stored at 2-8° C. The IVSS vial is stored at ambient temperature. The diluted active drug preparations have been tested at the lowest (100 ng/mL) and highest dose (160 μg/mL) with exposure to light for infusion set compatibility for up to 6 hours at controlled room temperature (20-25° C.) and for up to 12 hours at 2-8° C.

### Eastern Cooperative Oncology Group (ECOG) Performance Status:

Aspects of the invention include assessing an ECOG performance status of a subject at Screening, Day 1 of each cycle prior to dosing, at the EOT Visit or upon subject discontinuation, and at the 90-day follow-up visit after the last dose of TNB-383B. The ECOG performance status is documented using the scoring method in Table 8.

TABLE 8
Eastern Cooperative Oncology Group (ECOG) Performance Score

| Grade | ECOG |
|---|---|
| 0 | Fully active, able to carry on all pre-disease performance |
| | without restriction. |
| 1 | Restricted in physically strenuous activity but ambulatory |
| | and able to carry out work of a light or sedentary nature, |
| | eg, light housework, office work. |
| 2 | Ambulatory and capable of all self-care but unable to carry |
| | out any work activities. Up and about more than 50% of |
| | waking hours. |
| 3 | Capable of only limited self-care, confined to bed or chair |
| | more than 50% of waking hours. |
| 4 | Completely disabled. Cannot carry on any self-care. Totally |
| | confined to bed or chair. |

### Clinical Laboratory Tests:

Aspects of the invention include obtaining samples for the clinical laboratory tests outlined in Table 9, at a minimum at Screening (by a central laboratory), and locally at subsequent visits, the EOT Visit, and the 90-day follow-up visit.

A certified laboratory is utilized to process and provide results for the clinical laboratory tests. Laboratory reference ranges are obtained prior to the initiation of the study. The baseline laboratory test results for clinical assessment for a particular test are defined as the last measurement prior to the initial dose of TNB-383B.

TABLE 9
Clinical Laboratory Tests

| Laboratory | | |
|---|---|---|
| Test | Parameter | |
| Hematology | Hematocrit | Basophils (if detected; |
| | Hemoglobin | absolute count and %) |
| | RBC count | Eosinophils (if detected; |
| | WBC count | absolute count and %) |
| | Neutrophils (absolute | Platelet count (estimate |
| | count and %) | not acceptable) |
| | Bands (if detected) | MCH |
| | Lymphocytes (absolute | MCV |
| | count and %) | MCHC |
| | Monocytes (absolute | |
| | count and %) | |
| Coagulation | INR | aPTT |
| Panel | PT | |
| Serum | BUN | Serum pregnancy test |
| Chemistry | Creatinine | (where applicable) |
| | Total bilirubin | Total protein |
| | Albumin | Glucose |
| | AST | Chloride |
| | ALT | Bicarbonate/CO₂ |
| | Alkaline phosphatase | LDH |
| | Sodium | Magnesium |
| | Potassium | GGT |
| | Calcium | |
| Cytokinesₐ | IL-2 | IFN-gamma |
| | IL-6 | IL-1 |
| | TNF-alpha | IL-10 |
| T-Cell | T-Cell Subsets by | T-Cell Receptor |
| Evaluation | Flow Cytometry | Sequencing |
| Hepatitis | HBsAg | HCV Ab |
| B and C | | |
| (Screening | | |
| Only) | | |
| Urine | 24-hour urine collection for: | Protein |
| Tests | Total protein | Glucose |
| | UPEP with M-protein | Blood |
| | quantitation | Urobilinogen |
| | UIFE | Bilirubin |
| | Specific gravity | Microscopic |
| | Ketones | examination |
| | pH | |
| | Urine pregnancy test | |

TABLE 9
Clinical Laboratory Tests (cont.)

| Laboratory | | |
|---|---|---|
| Test | Parameter | |
| Myeloma- | Bone marrow aspiration/biopsy | SFLC |
| specific | with: | Quantitative immuno-globulins |
| | Immunohistochemistry and | (IgG, IgA, IgM; at baseline) |
| | quantitation of clonal plasma | Beta-2-microglobulin (at |
| | cell population | baseline) |
| | BCMA receptor density on | Exploratory: |
| | myeloma cells by standardized | [Soluble BCMA] in serum by |
| | flow cytometry | ELISA |
| | Cytogenetics and FISH analysis | [APRIL] in serum |
| | SIFE | |
| | SPEP with M-protein quantitation | |

Abbreviations: Ab = antibody; APRIL = a proliferation-inducing ligand; aPTT = activated partial thromboplastin time; ALT = alanine aminotransferase; AST = aspartate aminotransferase; BCMA = B cell maturation antigen; BUN = Blood urea nitrogen; ELISA = enzyme linked immunosorbent assay; FISH = fluorescence in situ hybridization; GGT = gamma-glutamyltransferase; HBsAg = hepatitis B surface antigen; HCV = hepatitis C virus; IFN = interferon; IL = interleukin; LDH = lactate dehydrogenase; MCH = mean corpuscular hemoglobin; PT = prothrombin time; MCHC = mean corpuscular hemoglobin concentration; MCV = mean corpuscular volume; RBC = red blood cell; SFLC = serum free light chains; SIFE = serum immunofixation electrophoresis; SPEP = serum protein electrophoresis; TNF = tumor necrosis factor; UIFE = urine immunophoresis electrophoresis; UPEP = urine protein electrophoresis; WBC = white blood cell;
^{a} The enumerated cytokines will be the minimally evaluated cytokines; analysis of additional cytokines in the samples submitted for cytokine analysis may be performed.

### Tumor Assessments:

Aspects of the invention involve performing a baseline skeletal survey with positron emission tomography (PET)-CT, contrast enhanced CT, or magnetic resonance imaging (MRI) for each patient within 28 days prior to administration of the first dose of TNB-383B; CT and/or MRI may also be performed for extramedullary disease assessment if clinically indicated. Imaging is repeated as clinically indicated. The same modality should be used for a subject at each visit where imaging is required, if possible.

### Tumor Tissue:

Aspects of the invention involve collecting, at screening, adequate archival tumor tissue (collected within the past 6 months prior to screening) and/or newly obtained biopsies for each subject; if no archival tumor tissue is available, a pre-treatment bone marrow biopsy is taken in some embodiments. An “adequate” archival biopsy is defined as sufficient formalin-fixed paraffin embedded (FFPE) material (either block or slides) to perform and interpret 8 to 10 hematoxylin and eosin and/or immunohistochemical stains on the subject's tumor and accompanied by a flow-cytometric report including plasma cell markers (at least CD38 and CD138). In some embodiments, tumor samples are analyzed at the molecular and cellular level to determine how baseline biomarker levels and changes from baseline relate to clinical outcomes, safety and resistance. In some embodiments, a bone marrow biopsy/aspirate for additional exploratory biomarker analyses is performed at cycle 3 day 1 (C3D1), in addition to IMWG mandated biopsies/aspirates at suspected CR, and when possible at suspected progression.

In some embodiments, both pre-treatment and progression biopsies are taken from the same lesion, or at least from the same anatomical location, of a patient. Collection of paired newly obtained tumor samples is used to assess TNB-383B PD in the tumor microenvironment. In some embodiments, the bone marrow biopsies are analyzed by flow cytometry to quantitate BCMA density on the subject's tumor cells. In some embodiments, studies such as cytogenetics, fluorescence in situ hybridization (FISH), or sequencing studies of tumor cells, and evaluation of T-cell subsets by flow cytometry are also performed. In some embodiments, the pre-treatment and suspected CR biopsies provide correlative data between tumor response and exploratory biomarkers (e.g., BCMA expression level), while the biopsies at C3D1 and suspected progression elucidate mechanisms of tumor resistance, if present and detectable. In some embodiments, tumor-specific deoxyribonucleic acid (DNA) alterations are investigated if it is found that a subset of subjects responds to therapy. In certain embodiments, this approach clarifies whether any genetic determinants are associated with responses. In addition, since anti-tumor immune responses may be related to the somatic mutation burden, the tumor mutation load is examined in certain embodiments.

### Sample Analysis:

In some embodiments, blood and/or tissue samples are collected from subjects at designated time points to evaluate PK, PD and response biomarkers as well as ADA. In some embodiments, serum samples for exploratory biomarker analysis (e.g., soluble BCMA and APRIL) are collected from subjects. In some embodiments, samples for exploratory biomarker analysis (e.g., soluble BCMA, APRIL, CRS-related cytokines, TCR sequencing, and T-cell subsets) are collected from subjects. In some embodiments, whole blood samples for DNA and RNA isolation are collected prior to dosing on Cycle 1 Day 1.

### Activity, Pharmacokinetic, Pharmacodynamic, Pharmacogenetic and Safety Assessments/Variables:

### Activity Variables:

In some embodiments, activity is measured by changes in SPEP, UPEP, and/or FLC (Kumar S et al., International Myeloma Working Group consensus criteria for response and minimal residual disease assessment in multiple myeloma, The Lancet Oncology. 2016; 17(8):e328-46). In some embodiments, the same parameter(s) used to meet eligibility criteria for treatment are used to evaluate response. In some embodiments, a laboratory value that indicates clinical activity (Table 10) is verified by a second test, which may be conducted as soon as the results from the first test are available. In some embodiments, activity endpoints (determined using IMWG uniform response criteria) include objective response rate (ORR, defined as stringent complete response [sCR]+complete response [CR]+very good partial response [VGPR]+partial response [PR]), clinical benefit rate (CBR; defined as CR+PR+MR for 24 weeks), overall survival (OS), progression-free survival (PFS), time to progression (TTP), time to response (TTR) and duration of objective response (DOR).

TABLE 10
Activity Measurements
Response criteria [symbol]
IMWG MRD criteria (requires a complete response as defined below)

| | |
|---|---|
| Sustained MRD-negative | MRD negativity in the marrow (NGF or NGS, or both) and by imaging as |
| | defined below, confirmed minimum of 1 year apart. Subsequent |
| | evaluations can be used to further specify the duration of negativity |
| | (eg, MRD-negative at 5 years) [symbol] |
| Flow MRD-negative | Absence of phenotypically aberrant clonal plasma cells by NGF [symbol] on bone |
| | marrow aspirates using the EuroFlow standard operation procedure for |
| | MRD detection in multiple myeloma (or validated equivalent method) |
| | with a minimum sensitivity of 1 in 10 [symbol] nucleated cells or higher |
| Sequencing MRD-negative | Absence of clonal plasma cells by NGS on bone marrow aspirate in which |
| | presence of a clone is defined as less than two identical sequencing reads |
| | obtained after DNA sequencing of bone marrow aspirates using the |
| | LymphoSIGHT platform (or validated equivalent method) with a |
| | minimum sensitivity of 1 in 10 [symbol] nucleated cells [symbol] or higher |
| Imaging plus MRD- | MRD negativity as defined by NGF or NGS plus disappearance of every |
| negative | area of increased tracer uptake found at baseline or a preceding PET/CT |
| | or decrease to less than the mediastinal blood pool SUV or decrease to less than |
| | that of surrounding normal tissue [symbol] |

Standard IMWG response criteria

[symbol]

| | |
|---|---|
| Stringent complete response | Complete response as defined below plus normal FLC ratio [symbol] and absence |
| | of clonal cells in bone marrow biopsy by immunohistochemistry (κ/λ ratio ≤4:1 |
| | or ≥1:2 for κ and λ patients, respectively, after counting ≥100 |
| | plasma cells) [symbol] |
| Complete response | Negative immunofixation on the serum and urine and disappearance of |
| | any soft tissue plasmacytomas and <5% plasma cells in bone marrow |
| | aspirates |
| Very good partial response | Serum and urine M-protein detectable by immunofixation but not on |
| | electrophoresis or ≥90% reduction in serum M-protein plus urine |
| | M-protein level <100 mg per 24 h |
| Partial response | ≥50% reduction of serum M-protein plus reduction in 24 h urinary |
| | M-protein by ≥90% or to <200 mg per 24 h: |
| | If the serum and urine M-protein are unmeasurable, a ≥50% decrease in |
| | the difference between involved and uninvolved FLC levels is required in |
| | place of the M-protein criteria; |
| | If serum and urine M-protein are unmeasurable, and serum-free light |
| | assay is also unmeasurable, ≥50% reduction in plasma cells is required in |
| | place of M-protein, provided baseline bone marrow plasma-cell |
| | percentage was ≥30%. In addition to these criteria, if present at baseline, |
| | a ≥50% reduction in the size (SPD) [symbol] of soft tissue plasmacytomas is also |
| | required |
| Minimal response | ≥25% but ≤49% reduction of serum M-protein and reduction in 24-h |
| | urine M-protein by 50-89%. In addition to the above listed criteria, if |
| | present at baseline, a ≥50% reduction in the size (SPD) [symbol] of soft tissue |
| | plasmacytomas is also required |
| Stable disease | Not recommended for use as an indicator of response; stability of disease |
| | is best described by providing the time-to-progression estimates. Not |
| | meeting criteria for complete response, very good partial response, partial |
| | response, minimal response, or progressive disease |
| Progressive disease [symbol] | Any one or more of the following criteria: |
| | Increase of 25% from lowest confirmed response value in one or more of |
| | the following criteria: |
| | Serum M-protein (absolute increase must be ≥0.5 g/dL); |
| | Serum M-protein increase ≥1 g/dL, if the lowest M component was ≥5 g/dL; |
| | Urine M-protein (absolute increase must be ≥200 mg/24 h); |
| | In patients without measurable serum and urine M-protein levels, the |
| | difference between involved and uninvolved FLC levels (absolute |
| | increase must be >10 mg/dL); |
| | In patients without measurable serum and urine M-protein levels and |
| | without measurable involved FLC levels, bone marrow plasma-cell |
| | percentage irrespective of baseline status (absolute increase must |
| | be ≥10%); |
| | Appearance of a new lesion(s), ≥50% increase from nadir in SPD [symbol] |
| | of >1 lesion, or ≥50% increase in the longest diameter of a previous |
| | lesion >1 cm in short axis; ≥50% increase in circulating plasma cells |
| | (minimum of 200 cells per μL) if this is the only measure of disease |
| Clinical relapse | Clinical relapse requires one or more of the following criteria: |
| | Direct indicators of increasing disease and/or end organ dysfunction |
| | (CRAB features) related to the underlying clonal plasma-cell proliferative |
| | disorder. It is not used in calculation of time to progression or |
| | progression-free survival but is listed as something that can be reported |
| | optionally or for use in clinical practice; |
| | Development of new soft tissue plasmacytomas or bone lesions |
| | (osteoporotic fractures do not constitute progression); |
| | Definite increase in the size of existing plasmacytomas or bone lesions. A |
| | definite increase is defined as a 50% (and ≥1 cm) increase as measured |
| | serially by the SPD [symbol] of the measurable lesion; |
| | Hypercalcemia (>11 mg/dL); |
| | Decrease in hemoglobin of ≥2 g/dL not related to therapy or other non- |
| | myeloma-related conditions; |
| | Rise in serum creatinine by 2 mg/dL or more from the start of the therapy |
| | and attributable to myeloma; |
| | Hyperviscosity related to serum paraprotein |
| Relapse from complete | Any one or more of the following criteria: |
| response (to be used only if | Reappearance of serum or urine M-protein by immunofixation or |
| the end point is disease-free | electrophoresis; |
| survival) | Development of ≥5% plasma cells in the bone marrow; |
| | Appearance of any other sign of progression (ie, new plasmacytoma, lytic |
| | bone lesion, or hypercalcemia see above) [symbol] |
| Relapse from MRD | Any one or more of the following criteria: |
| negative (to be used only if | Loss of MRD negative state (evidence of clonal plasma cells on NGF or |
| the end point is disease-free | NGS, or positive imaging study for recurrence of myeloma); |
| survival) | Reappearance of serum or urine M-protein by immunofixation or |
| | electrophoresis; |
| | Development of ≥5% clonal plasma cells in the bone marrow; |
| | Appearance of any other sign of progression (ie, new plasmacytoma, lytic |
| | bone lesion, or hypercalcemia) |

Abbreviations: ASCT = autologous stem cell transplantation; CRAB features = calcium elevation, renal failure, anemia, lytic bone lesions; [symbol] F-FDG PET = [symbol] F-fluorodeoxyglucose PET; FCM = flow cytometry; FLC = free light chain; IMWG =International Myeloma Working Group; MFC = multiparameter flow cytometry; M-protein = myeloma protein; MRD = minimal residual disease; NGF = next-generation flow; NGS = next-generation sequencing; SPD = sum of the products of the maximal perpendicular diameters of measured lesions; SUVmax = maximum standardized uptake value. [symbol]
indicates data missing or illegible when filed

### Pharmacokinetic Variables:

Aspects of the methods involve determining values for the PK parameters of TNB-383B, including the maximum observed serum concentration (Cmax), the time to Cmax (Tmax), the area under the concentration-time curve from time 0 to the time of the last measurable concentration (AUCt), clearance (CL), the terminal phase elimination rate constant (β), and terminal half-life (t_{1/2}) after infusion in cycle 1 using non-compartmental methods. In some embodiments, results of the ADA assays are analyzed post hoc.

### Drug Concentration Measurements:

### Collection of Blood Samples for TNB-383B Assay:

In some embodiments, blood is drawn from patients at one or more specified time points, and is used for TNB-383B PK analysis. Non-limiting examples of blood draw time points are listed below, and are also provided in Table 11 and Table 12.
Cycle 1: Day 1: pre-dose, at the end of the infusion and 3, 6, and 9 hours after the infusion; as well as on Days 2, 3, 8, and 15.
Cycle 2: Day 1: pre-dose, at the end of the infusion; as well as on Day 15.
Cycle 3: Day 1: pre-dose, at the end of the infusion, and 3, 6, and 9 hours after infusion; as well as on Day 15.
Cycle 4 and 5: Day 1: pre-dose and at the end of the infusion.
Cycle 6: Day 1: pre-dose, at the end of the infusion and 3, 6, and 9 hours after the infusion; as well as on Days 2 and 15.
Cycle 7 (and all subsequent cycles): Day 1: pre-dose and at the end of the infusion.
Unscheduled/End of Treatment (EOT) and 90 Day Follow-up
Cycle 1: Day 1: pre-dose, at the end of the infusion and 3, 6 and 9 hours after the infusion; as well as on Days 2, 3, 8, and 15.
Cycle 2: Day 1: pre-dose, at the end of the infusion; as well as on Day 15.
Cycle 3: Day 1: pre-dose, at the end of the infusion, and 3, 6, and 9 hours after infusion, as well as on Days 8 and 15.
Cycle 4 and 5: Day 1: pre-dose and at the end of the infusion.
Cycle 6: Day 1: pre-dose, at the end of the infusion and 3, 6, and 9 hours after the infusion; as well as on Days 2 and 15.
Cycle 7 (and all subsequent cycles): Day 1: pre-dose and at the end of the infusion.
End of Treatment, Suspected CR and 90 Day Follow-up

### Collection of Blood Samples for Antidrug Antibody (ADA) Assay:

In some embodiments, samples are analyzed for anti-drug antibodies. In some embodiments, additional ADA testing is conducted during PK sampling timepoints in the event of PK non-linearity. (See Table 11 and Table 12). In some embodiments, samples for ADA are drawn before dosing of TNB-383B on Day 1 of every cycle.

TABLE 11
Example schedule of blood sample collection (Arm A)

| | | | Post-Dose | | | | | | | | |
|---|---|---|---|---|---|---|---|---|---|---|---|
| | Pre- | | | | | 24 | | | | | |
| | Dose | EOI | 3 | 6 | 9 | hours ± | 48 | | ±1 day | | |
| | Within | ±10 | hours ± | hours ± | hours ± | 1 hour | hours ± | ±1 | Cycle | N/A | N/A |
| Dosing | 10 min | min | 10 min | 10 min | 1 hour | Cycle 1 | 1 hour | day | 1, 2, 3 | Unsched | 90-day |
| Cycle | All Cycles | | Cycle 1, 3, and 6 | | | and 6 | Cycle 1 | Cycle 1 | and 6 | Suspected | Follow |
| Day | Day 1 | | Day 1 | | | Day 2 | Day 3 | Day 8 | Day 15 | CR/EOT [symbol] | Up [symbol] |
| Study | | | | | | | | | | | |
| Activity [symbol] | | | | | | | | | | | |
| PK | X | X | X | X | X | X | X | X | X | X | X |
| Biomarker | X | X | | | | | | | | X | X |
| Assessments^{b} | | | | | | | | | | | |
| Anti-drug | X | | | | | | | | | X | X |
| Antibodies | | | | | | | | | | | |
| (ADAs) | | | | | | | | | | | |

| | |
|---|---|
| Cytokines [symbol] | See footnote c. |

Abbreviations: EOI = end of infusion: EOT = end of treatment; min = minute(s); N/A = not applicable; PK = pharmacokinetic(s); Unsched = unscheduled. [symbol]
Samples to be collected at the same time point will be included in a single blood draw. Blood samples for PK, biomarker assessments and ADA testing will be shipped to a central lab.
^{b}Testing for ADAs and biomarkers (soluble BCMA and APRIL) will be batch-analyzed centrally. Additional ADA testing will occur during PK sampling timepoints as appropriate. [symbol]
In the case of suspected cytokine release syndrome/neurotoxicity, cytokines will be collected per institutional guidelines and analyzed locally. [symbol]
Samples will be collected and batch-analyzed centrally for multiplex cytokines. In the case of suspected cytokine release syndrome/neurotoxicity, cytokines will be collected per standard of care and analyzed locally. [symbol]
One adequate-half-life data are available for TNB-383B and if T_{1/2} exceeds 18 days, the 90-day post-last treatment visit may be preformed at a time corresponding to ~5 × T_{1/2} in order to capture PK and ADA data at T ≥ 5 × T_{1/2}. [symbol]
An unscheduled visit may occur at any time during the study. Study activities shown will be performed at the investigator's discretion. [symbol]
indicates data missing or illegible when filed

TABLE 12
Example schedule of blood sample collection (Arm B)

| | Pre-Dose | | | Post-Dose | | | |
|---|---|---|---|---|---|---|---|
| | Within | | EOI | | | | 24 |
| | 10 min | | ±10 | 3 | 6 | 9 | hours ± |
| | Cycle | All | min | hours ± | hours ± | hour ± | 1 hour |
| Dosing | 1, 2, 3, | Other | All | 10 min | 10 min | 1 hour | Cycle |
| Cycle | and 6 | Cycles | Cycle | Cycle 1, 3, and 6 | | | 1 and 6 |
| Day | Day 1 | | | | | | Day 2 |
| Study Activity [symbol] | | | | | | | |
| PK | X | X | X | X | X | X | X |
| Biomarker | X | X | X | | | | |
| Assessments^{b} | | | | | | | |
| Pharmacogenomics [symbol] | X | X | | | | | |
| Anti-drug | X | X | | | | | |
| Antibodies (ADAs) | | | | | | | |
| Cytokines [symbol] | X | x: | X | X | x: | X | X |
| T-Cell Subsets | X | | | | | | |
| TCR Sequencing | X | | | | | | |

| | Post-Dose | | | | | |
|---|---|---|---|---|---|---|
| | | | ±1 | | | |
| | 48 | ±1 | day | | | |
| | hours ±1 | day | Cycle | | | |
| Dosing | hour | Cycle | 1, 2, 3 | N/A | N/A | N/A |
| Cycle | Cycle 1 | 1 and 3 | and 6 | Unsched/ | Suspected | Follow |
| Day | Day 3 | Day 8 | Day 15 | EOT [symbol] | CR | Up [symbol] |
| Study Activity [symbol] | | | | | | |
| PK | X | X | X | X | X | X |
| Biomarker | | | | X | X | |
| Assessments^{b} | | | | | | |
| Pharmacogenomics [symbol] | | | | | | |
| Anti-drug | | | | X | X | X |
| Antibodies (ADAs) | | | | | | |
| Cytokines [symbol] | X | X | X | X | X | |
| T-Cell Subsets | X | | X | | X | |
| TCR Sequencing | | | | | X | |

Abbreviations: EOI = end of infusion; min = minute(s); EOT = end of treatment; N/A = not applicable; PK = pharmacokinetic(s); Unsched = unscheduled. [symbol]
Samples to be collected at the same time point will be included in a single blood draw. All blood samples included in Table 2 will be shipped to the central lab.
^{b}Soluble BCMA and APRIL will be batched-analyzed centrally. [symbol]
As optional pharmacogenomics sample will be collected once the subject is deemed eligible for the study but before Cycle 1 Day 1 dosing. [symbol]
Samples will be collected and batch-analyzed centrally for multiplex cytokines. In the case of suspected cytokine release syndrome/neurotoxicity, cytokines will be collected per standard of care and analyzed locally. [symbol]
Once adequate half-life data are available for TNB-383B and if T_{1/2} exceeds 18 days, the 90-day post-last treatment visit may be performed at a time corresponding to −5 × T_{1/2} in order to capture PK data at T ≥ 5 × T_{1/2}. [symbol]
An unscheduled visit may occur at any time during the study. Study activities shown will be performed at the investigator's discretion. [symbol]
indicates data missing or illegible when filed

### Exploratory Research Variables:

In some embodiments, samples are collected to conduct exploratory investigations into known and novel biomarkers. The types of biomarkers to be analyzed may include, but are not limited to, nucleic acids, proteins, lipids or metabolites. In some embodiments, the samples are analyzed as part of a post hoc assessment of factors influencing the subjects' response to TNB-383B or the development and progression of the subjects' disease or related conditions. In some embodiments, the samples are used to develop new diagnostic tests, therapies, research methods or technologies.

### Safety Variables:

Aspects of the invention involve monitoring adverse events, laboratory profiles, physical exams, and vital signs of patients throughout treatment. In some embodiments, adverse events are graded according to the NCI-CTCAE, version 5.0. In some embodiments, standard PK, statistical, clinical, and laboratory procedures are utilized. In some embodiments, blood is drawn and analyzed for PD markers that may supply useful information in regard to selecting an appropriate dose of TNB-383B for additional treatment methodologies. In some embodiments, archival tissue is utilized in selecting an appropriate dose of TNB-383B for additional treatment methodologies, and for choosing an appropriate population of subjects for treatment.

### Determination of Maximum Tolerated Dose:

A maximum tolerated dose (MTD) is defined at the highest dose level at which less than 2 of 6 subjects experience a DLT. In some embodiments, an MTD is selected from the group consisting of: 25 μg, 75 μg, 200 μg, 600 μg, 1,800 μg, 5,400 μg, 10,000 μg, 20,000 μg, 30,000 μg, 40,000 μg, 50,000 μg, 60,000 μg, 70,000 μg, 80,000 μg, 90,000 μg, 100,000 μg, 110,000 μg, 120,000 μg, 130,000 μg, 140,000 μg, 150,000 μg, 160,000 μg, 170,000 μg and 180,000 μg.

### Determination of Recommended Phase 2 Dose:

If an MTD is reached, the RP2D (Recommended Phase 2 Dose) will not be a dose higher than the MTD, and will be selected based on the types of DLTs which occur and the MTD identified. If an MTD is not reached, then the RP2D will be defined based on the safety, PK and other available data.

### Dose Limiting Toxicity (DLT) Definition for Dose Escalation:

In some embodiments, the DLT observation period for dose escalation purposes is the first 21 days after the first dose of TNB-383B; dose limiting toxicities are determined on events that occur during the first observation period. Events occurring outside the DLT window may be evaluated when making dose escalation decisions. A drug related event is defined as any adverse reaction that cannot be definitively attributed to the patient's underlying disease, other medical condition, or a concomitant medication or procedure by the investigator or Medical Monitor. The NCI-CTCAE version 5.0 will be used. DLT definitions are provided herein.

### Non-Hematologic Dose-Limiting Toxicity:

A non-hematologic DLT is defined as any of the following TEAEs:
Non-hematological AEs Grade ≥3 with the following exceptions:
   Grade 3 or 4 isolated electrolyte abnormalities (i.e. those occurring without clinical consequence) that resolve, with or without intervention, to Grade <2 within 72 hours.
   Grade 3 hypo/hyperglycemia responsive to optimal medical management within 72 hours.
   Grade 3 nausea/vomiting/diarrhea responsive to optimal medical management within 72 hours.
   Alopecia or vitiligo of any grade.
   Grade 3 fatigue lasting <10 days.
Any AE that requires a delay in initiation of the next scheduled cycle by >21 days.

### Hematologic Dose-Limiting Toxicity

A hematologic DLT is defined as any of the following:
≥Grade 3 CRS.
Grade 4 neutropenia for >5 days.
Grade 3 thrombocytopenia requiring platelet transfusion or Grade 4 thrombocytopenia.
   During the course of the study, subjects with platelet counts <10,000/mm³ should be monitored at least every 72 hours (or more often at investigator's discretion) until recovery to >50,000/mm³. Platelet transfusions should be administered per Principal Investigator discretion and according to institutional guidelines. Recovery to >50,000/mm³ is required to continue treatment.
Grade 3 anemia associated with clinically significant symptoms of hypoxia requiring red blood cell transfusion or Grade 4 anemia, unrelated to underlying disease.
   Red cell transfusion in the absence of clinically significant hypoxic symptoms (i.e., tachypnea, decreased oxygen saturation, etc.) will not be considered a DLT.

Moderate anemia is not an exclusion criterion for subjects entering the study and is an anticipated finding in subjects with MM. Nevertheless, subjects with hemoglobin <8 g/dL should be monitored at least every 72 hours (or more often at investigator's discretion) until recovery to ≥8 g/dL. Red cell transfusions should be administered according to institutional guidelines. Recovery to ≥8 g/dL is required to continue treatment.
Lymphopenia will not be considered a DLT.
Any AE that requires a delay in initiation of the next scheduled cycle by >21 days.

### Cytokine Release Syndrome Dose-Limiting Toxicity:

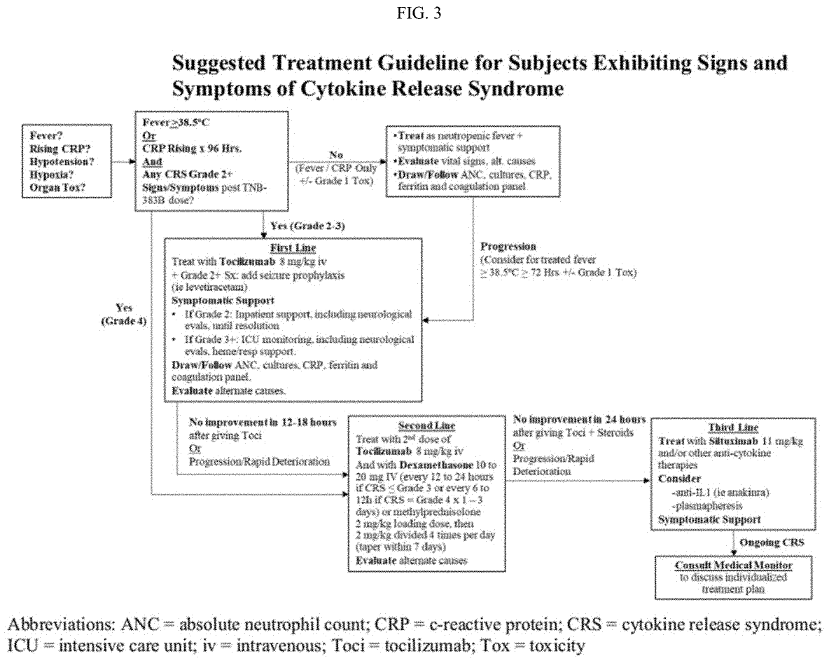

Aspects of the invention involve monitoring for evidence of cytokine release syndrome (CRS) in patients. Cytokine release syndrome (CRS), with or without the presence of neurotoxicity, is the primary toxicity associated with T-cell redirection therapy (CARs and T-BsAbs/BiTEs). CRS occurs due to hyper-activation of the immune system and is mediated predominantly by the secretion of pro-inflammatory cytokines (most importantly IL-6). Signs and symptoms are those of systemic inflammation, like sepsis, anaphylaxis and tumor lysis syndrome, and include the following: high fever/rigors, hypotension, hypoxia, neurologic changes, pain, nausea, and headache. Meta-analyses show that clinical findings, specifically fever, are usually the first indicators of CRS onset (Hay K A et al., Blood, Jan. 1, 2017; Wang Z et al., Biomarker Research. 2018; 6(1):4). CRS historically occurs within 14 days of first CAR/T-BsAb administration and does not usually occur in subsequent cycles. If CRS symptoms are suspected, Table 13 may be used to grade the toxicity and FIG. 3 provides a guideline for treatment that may be used for subject management, if an established treatment protocol is not in place.

TABLE 13
Guidelines for CRS Grading

| | CRS Grade 1 | CRS Grade 2 | CRS Grade 3 | CRS Grade 4 |
|---|---|---|---|---|
| Temperature >38.5 C. | Yes | Any | Any | Any |
| SBP ≤90 mmHg | No | Responsive | Requiring one | Life-threatening |
| | | to IV | pressor | |
| | | fluids | | |
| Supplement O₂ | No | FiO₂ <40% | FiO₂ >40% | Ventilator |
| for SO₂ <90% | | | | |
| Organ toxicity | Grade 1 | Grade 2 | Grade 3 | Grade 4 |
| | | | (with | |
| | | | increased | |
| | | | AST/ALT | |
| | | | Grade 4) | |

Source: CTCAE v 5.0

### Neurological Dose-Limiting Toxicity:

Aspects of the invention involve monitoring for evidence of neurological toxicity (NT). Neurological toxicity (NT) arises from unclear etiology but has been postulated to stem from endothelial activation/microangiopathy, possibly downstream of IL-1 secretion by monocytes/macrophages (Gust J et al., Cancer Discovery, 2017 Oct. 12; Giavridis T et al., Nature Medicine, 2018:1; Norelli M et al., Nature Medicine, 2018; 1). Onset usually occurs with or after CRS (mostly CRS Grade ≥3). Isolated NT has been described after administration of anti-CD19 T-BsAbs (Velasquez M P et al., Blood. 2017 Jan. 1). Symptoms of NT include: delirium, headache, agitation, aphasia, CNS bleed, ataxia, confusion, seizure, somnolence, and tremor. Suggested guidelines for management of NTs are provided in Table 14.

TABLE 14
Suggested Guidelines for Management of Neurological Toxicity

| AE Category | Guidelines |
|---|---|
| Neurologic AE after | Consult neurologist |
| dosing with TNB-383B | Continue neurologic monitoring |
| | Prophylactic anti-epileptic (i.e., levetiracetam) |
| | Consider MRI/CT, EEG, and/or LP |
| | Contact Medical Monitor |
| If the AE is Grade 2+, add | Consider Corticosteroids (e.g., dexamethasone 10 to 20 mg IV |
| | every 12 to 24 hours, higher doses or with more frequency, if |
| | clinically indicated) |
| If the AE is Grade 3+, add | High-dose corticosteroids (methylprednisolone 2 mg/kg loading |
| | dose followed by 2 mg/kg/day divided 4 times per day). |
| | Higher/more frequent doses if indicated. Taper when Grade 1 or |
| | better) |
| | Anti-epileptics for seizures/seizure-like activity |
| | ICU monitoring |
| | Airway protection |
| | Consider continuous EEG monitoring |
| If the AE is Grade 4+, add | If cerebral edema/other Grade 4 toxicity is rapid onset or the |
| | subjects is unresponsive to above: methylprednisolone IV |
| | 1 g/day × 3 days, followed if clinically indicated by taper. |
| | Consider hyperventilation/hyperosmolar therapy for cerebral |
| | edema. |

Abbreviations: AE = adverse event; CT = computed tomography; EEG = electroencephalogram; ICU = intensive care unit; IV = intravenous; LP = lumbar puncture; MRI = magnetic resonance imaging.

### Activity Analysis:

Aspects of the invention involve evaluating response and disease progression using IMWG uniform response criteria. In some embodiments, objective response rate (ORR), DOR, PFS and CBR are determined for both the Monotherapy Dose Escalation Phase (Arm A) and the Monotherapy Dose Expansion Phase (Arm B). In some embodiments, Kaplan-Meier estimates for PFS and associated CI of the median PFS, OS, and TTP are determined. For the Monotherapy Dose Expansion Phase (Arm B), activity analyses are performed, in some embodiments, based on the EE Population, and repeated for the Safety Population unless the sample size of the 2 populations is the same. In some embodiments, analyses from the Monotherapy Dose Escalation Phase (Arm A) for the MTD or RP2D are pooled with the Monotherapy Dose Expansion Phase (Arm B), as appropriate.

### Objective Response Rate (ORR):

Aspects of the invention involve determining an object response rate (ORR). Objective response rate is defined as the proportion of subjects with a confirmed partial or complete response to treatment. In some embodiments, the ORR for each dose cohort is estimated with all testing sites pooled. In some embodiments, the 2-sided 80% exact binomial CIs of ORR are also summarized using the Clopper-Pearson method along with the best overall response (CR, PR, SD, PD).

### Progression-Free Survival (PFS):

Aspects of the invention involve determining progression-free survival (PFS). Progression-free survival time is defined as the time from the first dose of TNB-383B to progression or death, whichever occurs first. In some embodiments, subjects are censored at the date of last tumor assessment if neither event occurs. In some embodiments, the Kaplan-Meier method will be used to analyze PFS.

### Duration of Objective Response (DOR):

Aspects of the invention involve determining a duration of objective response (DOR). The duration of objective response for a subject is defined as the time from the initial objective response to disease progression or death, whichever occurs first. In some embodiments, if a subject does not progress or die then the subject will be censored at the date of the last tumor assessment, similar to the censoring rules for the PFS analysis. In some embodiments, the DOR is analyzed in the same fashion as for the PFS analysis.

### Clinical Benefit Rate (CBR):

Aspects of the invention involve determining a clinical benefit rate (CBR). Clinical benefit rate is defined as the proportion of subjects with a confirmed complete, partial or minimal response for at least 24 weeks after responding to treatment. In some embodiments, the CBR for each arm is estimated with all testing sites pooled. In some embodiments, the 2-sided 80% exact binomial CIs of the CBR will also be summarized using the Clopper-Pearson method.

### Safety Analysis:

Aspects of the invention involve conducting one or more safety analyses. In some embodiments, at the end of a course of treatment, the safety of TNB-383B is assessed by evaluating the AEs, SUSARs/SAEs, changes in laboratory determinations, vital sign parameters and all other available relevant data. In some embodiments, the methods involve providing descriptive statistics for the continuous variables and the frequencies/percentages for the discrete variables. In some embodiments, the safety population allows for detection of SAEs occurring in as little as 21% of subjects with 80% confidence.

### Adverse Events:

Aspects of the invention involve analyzing adverse events, including, but not limited to, treatment emergent adverse events (TEAEs). A treatment emergent adverse event (TEAE) is defined as an event that occurs or worsens on or after the first dose of TNB-383B until 90 days following discontinuation of drug administration have elapsed, or until subjects start another anticancer therapy, whichever occurs earlier.

In some embodiments, TEAEs are summarized by dose cohort and overall including drug-related AEs, AEs by intensity, deaths, SAEs, and discontinuations due to AEs. In some embodiments, DLTs for the dose cohorts in the Monotherapy Dose Escalation Phase are summarized similarly by cohort and overall. In some embodiments, additional summaries and/or listings for AEs of special interest are also provided.

### Clinical Laboratory Tests:

Aspects of the invention involve performing baseline laboratory tests for patients receiving treatment. In some embodiments, disease response assessment laboratory tests from subsequent time points are also performed. In some embodiments, changes from baseline in clinical laboratory results are analyzed and summarized by dose cohort and time point using descriptive statistics. In some embodiments, summaries of shifts from baseline to last available visit are provided. In some embodiments, shifts are calculated as the proportion of subjects at baseline with values that are below, within, or above the normal range for a particular lab test, relative to the proportion of subjects at the Final Visit with values that are below, within, or above the normal range. In some embodiments, lab abnormalities and treatment-emergent lab abnormalities meeting the NCI-CTCAE version 5.0 are summarized by treatment arm and overall.

### Pharmacokinetics

### Tabulations and Summary Statistics

In some embodiments, serum concentrations of TNB-383B and PK parameter values are tabulated for each subject and each dose level, and summary statistics are computed for each sampling time and each parameter.

### Dose Proportionality Analysis:

In some embodiments, pharmacokinetic parameters of TNB-383B from a particular dosing schedule assessed on Cycle 1 Day 1 are analyzed as follows. An analysis is performed for dose-normalized Cmax and dose-normalized AUC. The model used for the statistical analyses includes the dose level of TNB-383B as a categorical variable. In some embodiments, covariates such as age, ethnicity, gender, and others that might explain some of the variability in the population are included in an initial model. In some embodiments, a covariate may be dropped from the model if the regression coefficient is not significant at alpha level 0.10. In some embodiments, a natural logarithmic transformation is employed for Cmax and the AUCs unless the data clearly indicate that other transformation or the untransformed variable provides more nearly symmetric probability distributions and/or more nearly homogenous variances across dose levels. When at least 3 TNB-383B dose levels are studied, a test is performed on a contrast in the dose level effects chosen to be sensitive to an approximately linear function of dose or the logarithm of dose.

### Missing Values and Model Violations

In some embodiments, all available data are included in any dose proportionality analyses. In some embodiments, one or more data points may be excluded from an analysis, provided an appropriate justification is present. Normally, values of PK variables (Cmax, AUC, etc.) are determined without replacing missing individual concentration values, by simply using the available data. However, if a missing individual concentration results in a PK parameter value that may be too low or too high to a meaningful degree, the value of the PK parameter may tentatively be considered missing. In such cases, a value for the missing individual concentration may be imputed so that an appropriate value of the PK parameter can be included in an analysis. In some embodiments, the imputed value is obtained using appropriate methodology that considers the individual characteristics of the subject.

If an outlier is identified and/or a pronounced non-normal probability distribution is observed (after logarithmic transformation for Cmax and AUC) then a non-parametric analysis may also be performed. Such a model violation may be identified by graphical methods, measures of non-normality (e.g., skewness, kurtosis) or other appropriate methods. If different dose levels have unequal variances to the extent that conclusions might be affected, then approximate methods that allow for unequal variances can be used. In some embodiments, the possibility of bias from missing data of subjects who prematurely discontinued treatment due to an adverse event can be addressed.

### Biomarkers:

Aspects of the invention involve exploratory biomarker analysis. In some embodiments, descriptive statistics of the baseline, post-baseline, and change from baseline of biomarkers are analyzed and summarized by measurement time point/visit. In some embodiments, exploratory analyses are performed to evaluate the association of each biomarker or combination of biomarkers with clinical outcomes, the modulation of biomarkers related to mechanism of action, and biomarker or combination of biomarkers potentially predictive of treatment response.

### EXAMPLES

### Example 1: Initial Results of a Phase I Study of TNB-383B in Relapsed/Refractory Multiple Myeloma

Background: TNB-383B is a BCMA×CD3 bispecific T-cell redirecting antibody incorporating a unique anti-CD3 moiety that preferentially activates effector over regulatory T-cells and uncouples cytokine release from anti-tumor activity, as well as 2 heavy-chain-only anti-BCMA moieties for a 2:1 TAA to CD3 stoichiometry. Results from the ongoing phase 1 dose escalation and expansion FIH study of TNB-383B are presented (NCT03933735).

Methods: Eligible patients have RRMM and have been exposed to at least 3 prior lines of therapy including a proteasome inhibitor (PI), an immunomodulatory drug (IMiD), and an anti-CD38 monoclonal antibody. Patients were treated with escalating doses of TNB-383B infused IV over 1-2 hours Q3W (without step-up dosing). The primary objectives were to determine the safety/tolerability and clinical pharmacology of TNB-383B and to identify the MTD/RP2D. The study used a 3+3 design for dose escalation, with additional patients enrolled on cleared dose levels. Patients on earlier dose cohorts were allowed to increase to the highest cleared dose levels. Responses were assessed by IMWG criteria and Adverse Events were graded according to CTCAE v5.0. Minimal residual disease (MRD) assessment was performed via NGS of bone marrow samples.

Results: 38 subjects were dosed with TNB-383B (0.025-40 mg). Demographics and disease characteristics at study entry are summarized in Table 15. The most common Gr3/4 AEs were Anemia (6/38; 16%) and Thrombocytopenia (5/38; 13%). The most common drug-related AEs were CRS (8/38; 21%) and Headache (5/38; 13%). An isolated case of Gr2 CRS was observed at 75 μg but all other CRS was seen at 5.4 mg and above. All cases of CRS were grade 1 (5/8) or 2 (3/8) and all occurred after the first dose of TNB-383B only. All but 3 subjects were managed with fluids and Tylenol (the other 3 received 1 dose of tocilizumab). One DLT, Gr3 Confusion that resolved within 6 hours without sequelae was seen at the 20 mg dose. No IRRs were observed. Dose modification was necessary in 1 subject for Gr 3 neutropenia associated with CRS; the subject was returned to their full dose after tolerating subsequent doses without incident. 5 subjects died from their underlying disease during follow-up. 15 subjects discontinued treatment, all of them for progressive disease. Preliminary PK data support Q3W dosing of TNB-383B. Activity was observed in one patient each at 200 μg and 1.8 mg; at doses of 5.4-20 mg an ORR of 55% (12/22) was observed. Depth and duration of response are summarized in Table 16.

Conclusions: TNB-383B is well tolerated at doses up to 40 mg without the need for Step/Split Dosing. A preliminary ORR of 55% was observed at doses ≥5.4 mg, including deep (3 VGPR/3 CR) and durable (up to 24 weeks) responses despite dosing only every 3 weeks.

TABLE 15
Summary of patient demographics and disease characteristics

| | 0.025-1.8 mg | | >5.4 mg | | Total | |
|---|---|---|---|---|---|---|
| Subjects | N = 15 | | N = 23 | | N = 38 | |
| Male | 10 | (67%) | 11 | (48%) | 21 | (55%) |
| Female | 5 | (33%) | 12 | (52%) | 17 | (45%) |
| Median Age (Range) | 72 | (56-83) | 68 | (37-78) | 68 | (37-83) |
| Median Prior Lines of | 8 | (4-12) | 7 | (4-13) | 7 | (4-13) |
| Therapy (Range) | | | | | | |

TABLE 16
Response Summary

| | 0.025-1.8 mg | | >5.4 mg | | Total | |
|---|---|---|---|---|---|---|
| Subjects | N = 15 | | N = 23 | | N = 38 | |
| ORR | 2 | (13%) | 12 | (52%) | 14 | (37%) |
| sCR/CR | 0 | | 3 | (13%) | 3 | (7.9%) |
| VGPR | 1 | (6.7%) | 3 | (13%) | 4 | (11%) |
| PR | 1 | (6.7%) | 6 | (26%) | 7 | (18%) |
| Median DOR in Weeks | 24 | (21-27) | 9 | (3-21) | 9 | (3-27) |
| (Range) | | | | | | |

### Example 2: Further Initial Results of a Phase I Study of TNB-383B in Relapsed/Refractory Multiple Myeloma

Background: TNB-383B is a BCMA×CD3 bispecific T-cell redirecting antibody incorporating a unique anti-CD3 moiety that preferentially activates effector over regulatory T-cells and uncouples cytokine release from anti-tumor activity, as well as 2 heavy-chain-only anti-BCMA moieties for a 2:1 TAA to CD3 stoichiometry. Results from the ongoing phase 1 dose escalation and expansion FIH study of TNB-383B are presented (NCT03933735).

Methods: Eligible patients have RRMM and have been exposed to at least 3 prior lines of therapy including a proteasome inhibitor (PI), an immunomodulatory drug (IMiD), and an anti-CD38 monoclonal antibody. Patients were treated with escalating doses of TNB-383B infused IV over 1-2 hours Q3W (without step-up dosing). The primary objectives were to determine the safety/tolerability and clinical pharmacology of TNB-383B and to identify the MTD/RP2D. The study used a 3+3 design for dose escalation, with additional patients enrolled on cleared dose levels. Patients on earlier dose cohorts were allowed to increase to the highest cleared dose levels. Responses were assessed by IMWG criteria and Adverse Events were graded according to CTCAE v5.0. Minimal residual disease (MRD) assessment was performed via NGS of bone marrow samples.

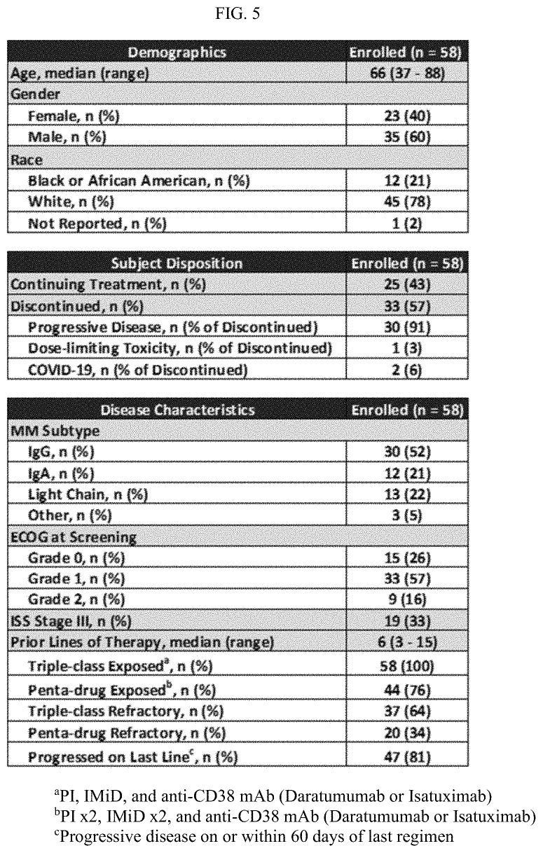

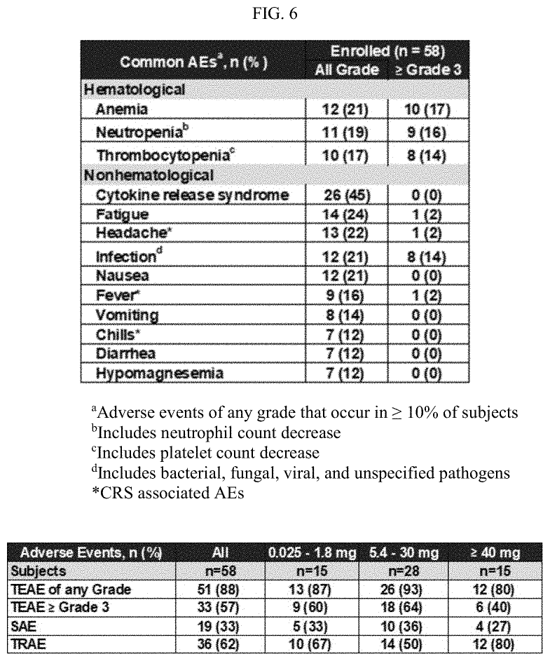

Results: 58 subjects were dosed with TNB-383B (0.025-60 mg). Demographics and disease characteristics at study entry are summarized in FIG. 5. The most common AEs observed are summarized in FIG. 6. Cytokine release syndrome (CRS) was the most common AE on study, affecting 45% of subjects. Hematologic toxicities were seen in approximately 20% of all subjects. The most common grade 3 or 4 non-hematologic adverse event was infection in 14% of patients. These infections were similar in character and duration to those previously described in the relapsed/refractory myeloma population. Other grade 3 or higher AEs were rare. While treatment-related AEs increased due to CRS at doses of 40 mg or higher, non-CRS AEs, including those of Grade 3 or 4, have remained stable to date. There have been 2 dose limiting toxicities observed thus far: a Grade 3 confusion not associated with ICANS and believed to be related to transient fever, and a Grade 4 thrombocytopenia that recovered to normal levels by the end of the cycle.

No marked increase in the incidence of non-CRS AEs was observed with higher doses of TNB-383B. TRAE increased at doses greater than or equal to 40 mg due to increased CRS. As reviewed above, two DLTs were observed, and both resolved without sequelae. These included Gr3 confusion at 20 mg (not ICANS-related) and Gr4 thrombocytopenia at 60 mg. Two deaths occurred on study, both due to COVID-19 (not study drug-related).

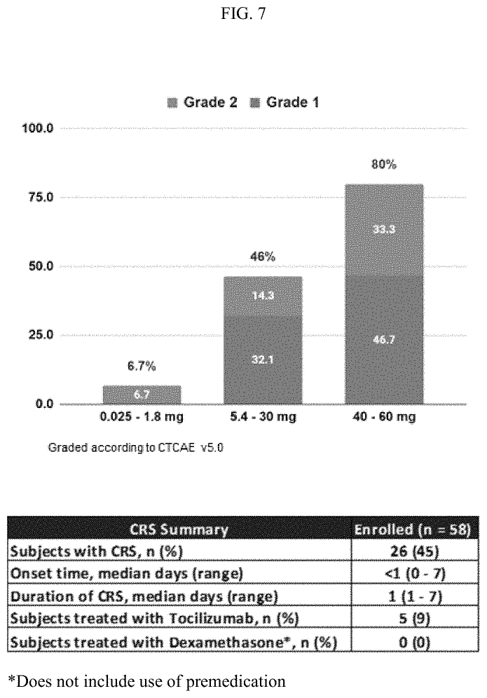

FIG. 7 provides a summary of CRS as a function of dose. As noted above, 45% of treated patients experienced some degree of CRS. At doses of 40 mg or higher, 80% of patients had CRS. However, all CRS cases were limited to grade 1 and 2, with no cases of grade 3 CRS or higher at any of the doses given. Furthermore, split-dosing was not required at any dose.

Subjects that did experience CRS were managed predominantly with fluids and acetaminophen. Only five patients received tocilizumab for their symptoms per PI discretion. No subjects received dexamethasone as treatment for CRS. CRS onset usually occurred within 24 hours of dosing with a median duration of 1 day. Interestingly, none of the patients who underwent dose-escalation developed CRS at the increased dose, even though escalations of up to 6-fold were implemented.

As noted above, minimally worsening severity of CRS was observed with increased doses of TNB-383B. Re-occurrence of CRS post-C1 was observed in only 1 subject, and no CRS was observed in subject undergoing intra-patient dose escalation. Step-/split-dosing of TNB-383B was not required, and study drug was administered as a bolus at all dose levels.

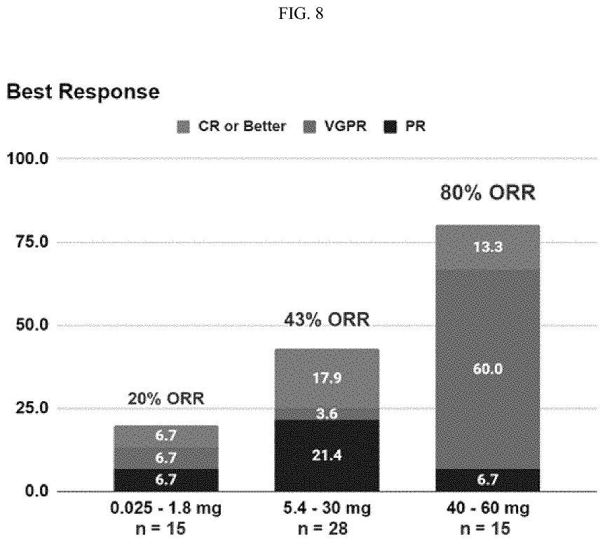

FIG. 8 is a graph summarizing response to therapy. Overall response rate (ORR) in those patients receiving treatment at 40-60 mg dosing was 80%. Of those, more than 70% achieved a VGPR or better. Three of four MRD-evaluable subjects were MRD-Negative (2 at 10⁶, 1 at 10⁵). Activity was observed in one patient at doses as low as 200 micrograms. The responses were significantly improved in the 15 patients treated at higher doses compared to smaller doses. The depth of response seen in the higher doses stands out from the rest of the study patients, with 60% achieving a VGPR and 13% achieving a CR or better.

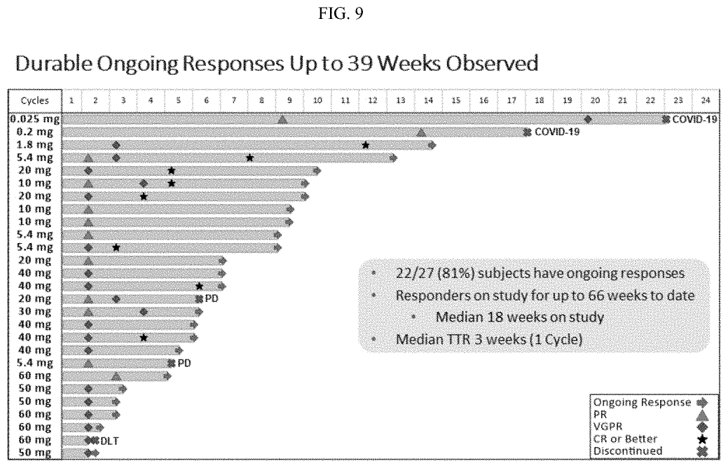

FIG. 9 is a swimmer plot showing the trajectory of the 27 patients who have responded to treatment while on study. The median time on therapy among responders at data cut-off was 18 weeks, with the longest time on therapy being 66 weeks. 22 of the 27 responders remain on therapy.

Of the responders that discontinued, 2 had progressive disease, 1 had a DLT of Grade 4 thrombocytopenia, and 2 died due to COVID-19 infection. In most cases, a significant response was seen with the first dose of TNB-383-B. In addition, responses deepened over time as patients remained on therapy.

### Example 3: Characterization of Pharmacokinetics (PK) of TNB-383B

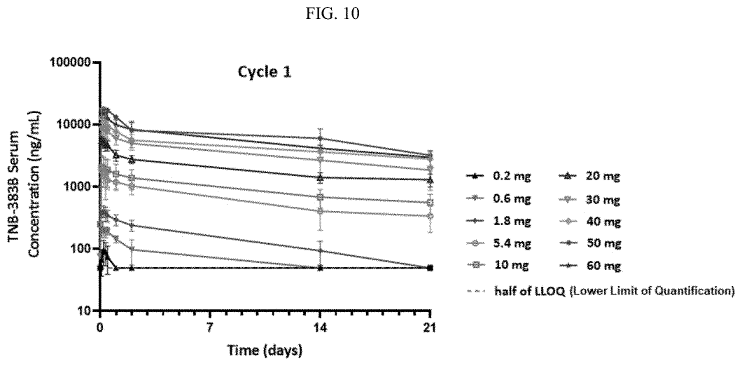

FIG. 10 is a graph of PK data for TNB-383B, which was characterized as a primary endpoint of the clinical study. The PK data available to date are shown in FIG. 10, and are consistent with typical antibody therapeutics. At doses up to 1.8 mg, the PK was not dose proportional, possibly due to target-mediated clearance. At doses of 5.4 mg and above, PK was approximately dose proportional, and at doses of 20 mg or more, TNB-383B showed a half-life of 15 to 18 days, supporting a dosing schedule of once every 3 weeks.

### Example 4: Case Study

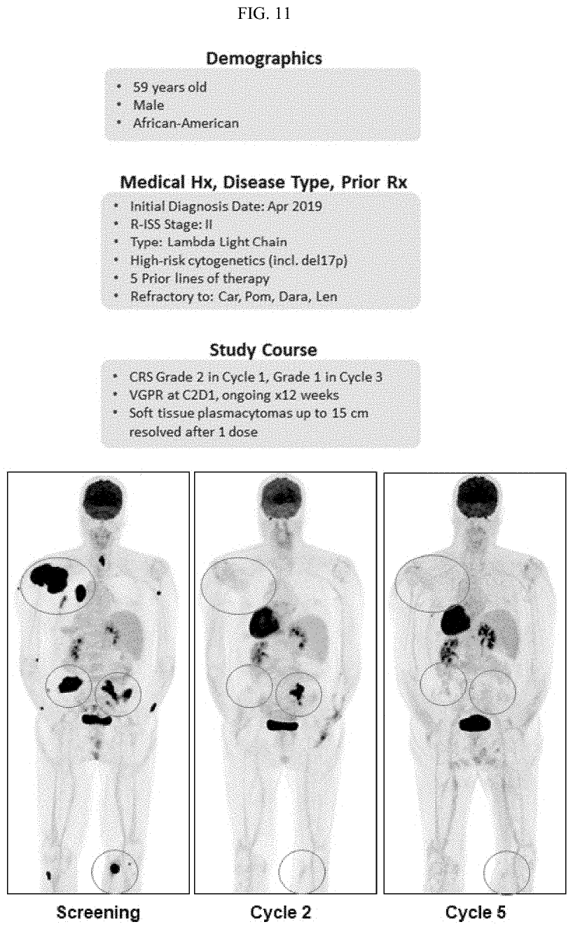

A case study analysis of a 59 year old African American male subject with high-risk cytogenetics, and who was refractory to IMiDs, PIs, and monoclonal antibodies, is presented in FIG. 11. This subject had extensive plasmacytomas prior to study enrollment, as evidenced by the Screening image panel in FIG. 11. This subject achieved a VGPR by serology after 1 dose of therapy. Repeat imaging after 2 and 5 treatment doses showed a dramatic reduction in his extramedullary plasmacytomas, as seen in the Cycle 2 and Cycle 5 panels. The subject remains on therapy with an ongoing VGPR.

### Example 5: Study Conclusions to Date

Conclusions thus far obtained from the clinical study of TNB-383B include the following: TNB-383-B is a novel bi-specific T-cell engaging immunotherapy targeting BCMA and CD3 that is well tolerated at all tested doses with few off-target toxicities. This off-the-shelf BCMA-targeted therapy has been given safely in the office setting after a short hospitalization following administration of the first dose. To date, no CRS grade 3 or higher has been seen at any dose, and step-up dosing has not been required.

An ORR of 80% at doses of 40 mg and higher, with a high number of VGPR or better responses was achieved despite a patient population with multiple prior lines of therapy. With its safety profile, efficacy, and convenience of once every 3-week dosing, this agent makes for a promising option for myeloma treatment. Escalation and expansion portions of this study are ongoing.

1. A method for treating multiple myeloma (MM) in a patient in need, the method comprising administering to the patient a therapeutically effective amount of TNB-383B according to a 21-day treatment cycle, wherein the therapeutically effective amount of TNB-383B is a flat dose ranging from 10 mg to 100 mg.

2. The method according to claim 1, wherein the treatment cycle is repeated two or more times.

3. The method according to claim 1, wherein TNB-383B is administered to the patient as a monotherapy.

4. The method according to claim 1, wherein TNB-383B is administered by an intravenous infusion (IV).

5. The method according to claim 1, wherein the MM is relapsed MM.

6. The method according to claim 1, wherein the MM is refractory MM.

7. The method according to claim 1, wherein the patient has received at least three prior lines of therapy.

8. The method according to claim 7, wherein one of the prior lines of therapy comprises treatment with a proteasome inhibitor (PI).

9. The method according to claim 7, wherein one of the prior lines of therapy comprises treatment with an immunomodulatory imide (IMiD).

10. The method according to claim 7, wherein one of the prior lines of therapy comprises treatment with an anti-CD38 antibody.

11. The method according to claim 10, wherein the anti-CD38 antibody is a monoclonal antibody.

12. The method according to claim 11, wherein the anti-CD38 monoclonal antibody is daratumumab.

13. The method according to claim 1, where in the patient is not a candidate for one or more treatment regimens that are known to provide a clinical benefit in MM.

14. A method for improving an objective response rate (ORR) in a patient diagnosed with MM, the method comprising administering to the patient a therapeutically effective amount of TNB-383B according to a 21-day treatment cycle, wherein the therapeutically effective amount of TNB-383B is a flat dose ranging from 10 mg to 100 mg.

15.-25. (canceled)

26. The method according to claim 1, wherein the treatment cycle is modified to increase a dosing frequency.

27. The method according to claim 26, wherein the treatment cycle is modified by reducing the time between doses to 14 days.

28.-29. (canceled)

30. The method according to claim 1, further comprising premedicating the patient prior to administration of TNB-383B with an agent that reduces a risk or severity of a hypersensitivity reaction.

31. The method according to claim 30, wherein the agent that reduces the risk or severity of a hypersensitivity reaction is selected from the group consisting of: dexamethasone, diphenhydramine, acetaminophen, ranitidine, any equivalents thereof, or any combination thereof.

32. The method according to claim 30, wherein the agent that reduces the risk or severity of a hypersensitivity reaction is administered 15 to 60 minutes prior to administration of TNB-383B.

33. (canceled)

34. A method for treating relapsed or refractory multiple myeloma in a patient in need, the method comprising administering TNB-383B to the patient at a flat dose ranging from 10 mg to 100 mg, administered once every 3 weeks (21 days), wherein the patient has received at least three prior lines of therapy, including a proteasome inhibitor (PI), an immunomodulatory imide (IMiD) and an anti-CD38 monoclonal antibody (mAb).

35. The method of claim 34, wherein the flat dose of TNB-383B is 60 mg.

36. The method of claim 1, wherein the flat dose of TNB-383B is 60 mg.

37. The method of claim 14, wherein the flat dose of TNB-383B is 60 mg.
